(19) United States
(12) Patent Application Publication
(10) Pub. No.: US 2013/0071326 A1
(43) Pub. Date: Mar. 21, 2013

(54) UNIVERSAL CELL-DIRECTED THERANOSTICS

(71) Applicants: Jonathan O. Martinez, Houston, TX (US); Ennio Tasciotti, Houston, TX (US); Mikhail Kolonin, Houston, TX (US); Mauro Ferrari, Houston, TX (US)

(72) Inventors: Jonathan O. Martinez, Houston, TX (US); Ennio Tasciotti, Houston, TX (US); Mikhail Kolonin, Houston, TX (US); Mauro Ferrari, Houston, TX (US)

(73) Assignee: Board of Regents of the University of Texas System, Austin, TX (US)

(21) Appl. No.: 13/635,592

(22) PCT Filed: Mar. 17, 2011
(86) PCT No.: PCT/US11/28890

§ 371 Date: Nov. 27, 2012

Related U.S. Application Data
(60) Provisional application No. 61/282,688, filed on Mar. 17, 2010.
(60) Provisional application No. 61/282,691, filed on Mar. 17, 2010.

(51) Int. Cl. A61K 35/12; C12N 5/074; C12N 5/0775; C12N 5/0735; C12N 5/0797; A61K 35/30; A61K 35/54; A61P 29/00; A61P 35/00; A61P 17/02; A61K 9/127; A61K 9/70; A61K 49/06; A61K 49/04; A61K 51/06; A61K 49/22; C12N 5/071; B82Y 5/00

(52) U.S. Cl. 424/1.73; 435/325; 435/375; 424/93.7; 424/450; 424/400; 424/9.3; 424/9.4; 424/9.5; 977/773; 977/906

(57) ABSTRACT

The present invention provides modified stem cells that comprise a delivery system that comprises at least one microparticle or nanoparticle, wherein the at least one microparticle or nanoparticle comprises an active agent. The present invention also provides delivery methods that comprise the administration of the modified stem cells to a subject. Additional aspects of the present invention pertain to methods of making said modified stem cells.

### CROSS-REFERENCE TO RELATED APPLICATIONS

This application claims priority to U.S. Provisional Patent Application Nos. 61/282,688 and 61/282,691, both filed on Mar. 17, 2010. This application is also related to PCT/US11/28861, filed on Mar. 17, 2011. The entirety of each of the above-identified applications are incorporated herein by reference.

### STATEMENT REGARDING FEDERALLY SPONSORED RESEARCH

This invention was made with government support under Grant No. NNJ06HE06A, awarded by the National Aeronautics and Space Administration; Grant No. W81XWH-07-2-0101, awarded by the U.S. Department of Defense; and DARPA Grant No. W911NF-09-1-0044, also awarded by the U.S. Department of Defense. The government has certain rights in the invention.

### BACKGROUND OF THE INVENTION

Systems and compositions for delivering active agents to desired sites in organisms have numerous therapeutic, preventive, imaging, and diagnostic applications. Current systems and compositions for achieving such tasks suffer from numerous limitations, including specificity and efficacy. Therefore, there is currently a need to develop more effective systems and compositions for delivering active agents to desired sites in organisms

### BRIEF SUMMARY OF THE INVENTION

In some embodiments, the present invention provides modified stem cells that comprise a delivery system. The delivery system comprises at least one microparticle or nanoparticle that further comprises an active agent (e.g., a therapeutic agent and/or imaging agent). In some embodiments, the microparticle or nanoparticle is a porous particle, such as a nanoporous silicon particle. In some embodiments, the stem cell is an adipose stromal stem cell. In some embodiments, the delivery system is a multistage delivery system.

Further embodiments of the present invention pertain to methods of delivering the above-described modified stems cells to a subject. Additional embodiments of the present invention pertain to methods of making the above-described delivery systems.

The methods, systems and modified stem cells of the present invention have numerous applications and advantages. For instance, various aspects of the present invention may be utilized for the treatment and diagnosis of inflammatory disorders, such as inflammatory disorders associated with a cancer. Such modes of treatment and diagnosis are advantageously more specific and effective than the methods and systems of the prior art. For instance, in some embodiments, the modified stems cells of the present invention may be used to selectively deliver large payloads of both therapeutic and/or imaging agents or vectors to a desired tissue (e.g., inflamed tissues).

### BRIEF DESCRIPTION OF THE FIGURES

In order that the manner in which the above recited and other advantages and objects of the invention are obtained, a more particular description of the invention briefly described above will be rendered by reference to specific embodiments thereof, which are illustrated in the appended Figures. Understanding that these Figures depict only typical embodiments of the invention and are therefore not to be considered limiting of its scope, the invention will be described with additional specificity and detail through the use of the accompanying Figures in which:

FIG. 1 provides an overview of a method of making and delivering modified stem cells in accordance with various embodiments of the present invention. In this embodiment, the approach is used to target tumors with adipose-directed nanotherapeutics.

FIG. 1A shows the loading of therapeutic doxorubicin (DOX) and a diagnostic iron oxide-based imaging agent (SPIO) into a nanoporous silicon particle (NP). The NP is then loaded into a multistage nanoshuttle particle (MSN).

FIGS. 1B-1C show the internalization of the MSN's into adipose stromal stem cells (ASC).

FIG. 1D shows the ASCs carrying NP-loaded MSNs being subcutaneously (s.c.) administered into tumor bearing mice, where they can be imaged.

FIGS. 1E-1F show the NP-loaded MSN homing to the tumor site after administration.

FIG. 1G shows the dissociation of the MSNs from the ASCs after reaching the tumor site.

FIG. 1H shows the release of DOX from the MSNs, and the killing of the tumor cells by the released DOX.

FIG. 2 shows that the release of proteins, active agents and other biomolecules from nanoparticles can be tailored according to the surface coatings used. In the experiments, different polymers, alone or in combination, were conjugated on the surface of porous particles to prevent the burst release of the payload, and to achieve a controlled, sustained release. This data also indicates that the early release of DOX can be prevented through polymeric surface functionalization.

FIG. 3 shows studies investigating the therapeutic potential of ASC/MSN nanosystems.

FIG. 3A shows confocal images of ASC incubated with DOX-loaded MSN at a concentration of 1:15 (cells:MSN) and stained with Lysotracker (lysosomes).

FIG. 3B shows ASC viability (MTT assay) in the presence of MSN, where 1:20 and 1:40 concentrations are compared.

FIG. 3C shows flow cytometry analysis of the shape and fluorescence difference between ranges of concentrations (1:20 to 1:100).

FIG. 3D shows a two-week viability assay examining the growth of cells incubated with MSN loaded with NP-encapsulated DOX: no growth difference between control groups is seen until after the first week.

FIG. 3E shows fluorescence image showing the internalization of MSN with NP-DOX. ASC nuclei are blue. MSN are yellow. DOX is red

FIG. 3F shows that in vivo near infrared (NIR) whole-body optical images provide evidence for the capacity of ASC to deliver MSN/DOX to tumors. Detection of DOX in mice xenografted with a tumor (right shoulder) 1 hour post-injection (on both flanks) with ASC (left mouse) or with ASC carrying MSN with DOX-loaded NP (right mouse) demonstrates ASC migration toward the tumor.

FIG. 4 (right bracket) shows a flow cytomteric gating strategy used for isolation of ASC from mouse white adipose tissue (WAT) and their purification from WAT-resident adipose endothelial cells (AEC) and monocytes. The right panel shows phase contrast images of representative colonies (passage 1). The left bracket shows a flow cytomteric gating strategy used for isolation of ASC from mouse WAT and their purification from WAT-resident adipose endothelial cells (AEC) and monocytes. The images shown represent phase contrast images of representative colonies (passage 1).

FIG. 5 shows an example of ASC isolation from a patient WAT sample.

FIG. 5A shows a flow cytomteric gating strategy discriminating ASC from endothelial cell (EC), and hematopoietic/endothelial circulating progenitor cells (CPC).

FIG. 5B shows phase contrast images of the respective cell populations (passage 1).

FIG. 5C shows a fluorescent image of human ASC infected with lentivirus-GFP.

FIG. 6 shows that the loading of MSN with iron oxide NPs and chitosan-coated iron oxide NPs (cIONP) depend on the surface chemistry of MSNs. The SEM and TEM images show loading into the pores, and the endosomal escape of 30 nm cIO NPs 24 hours following cell internalization, respectively. The black arrows indicate cIO inside the endosome, while the white arrows point at cIO that escaped the endosome.

FIG. 7 establishes MDS compatibility within ASC.

FIGS. 7A-7B show that ASCs efficiently internalize MDS particles and gold nanoparticles (AuNPs), respectively. The bars are 100 nm scale bars.

FIG. 7C shows that the incorporation of MDS shows no affect on cytoskeletal structure. Actin (red), tubulin (green) and MDS (yellow) are shown.

FIG. 7D demonstrates that, when exposed to MDS particles carrying DOX encapsulated within micelles, DOX does not immediately distribute to ASC but remains inside the MDS.

FIG. 8 shows in vitro migration assays.

FIGS. 8A-8B show live microscopy snap shots after 18 hours of migration of ASC without particles (FIG. 8A) and with MSN (FIG. 8B).

FIGS. 8C-8D show confocal images after ASCs were allowed to migrate for 24 hours without MSNs (FIG. 8C), and with MSNs (FIG. 8D). ASCs are seen to migrate more than 500 μm in this time after transduction with firefly luciferase and mCherry (Orange) towards breast cancer cells expressing GFP (green). MSN particles are in yellow (FIG. 8D).

FIG. 9 shows in vitro differentiation of ASCs. The introduction of MSN (Si particles) did not interfere with the ASCs' ability to differentiate into fat and bone.

FIG. 10 shows validation of ASCs as vehicles. ASCs were labeled with a NIR dye and monitored using NIR imaging upon s.c. administration in breast tumor bearing mice. After three days, a large accumulation of fluorescence is found at the tumor site, as indicated by yellow arrows and confirmed using histology (black arrows indicate MDS).

### DETAILED DESCRIPTION OF EXEMPLARY EMBODIMENTS

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents. patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Related Applications and Publications

The following research articles and patent documents, which are all incorporated herein by reference in their entirety, may be useful for understanding the present inventions: (1) PCT Publication No. WO 2007/120248 (published on Oct. 25, 2007); (2) PCT Publication No. WO 2008/041970 (published on Apr. 10, 2008); (3) PCT Publication No. WO 2008/021908 (published on Feb. 21, 2008); (4) U.S. Patent Application Publication No. 2008/0102030 (published on May 1, 2008); (5) U.S. Patent Application Publication No. 2003/0114366 (published on Jun. 19, 2003); (6) U.S. Patent Application Publication No. 2008/0206344 (published on Aug. 28, 2008); (7) U.S. Patent Application Publication No. 2008/0280140 (published on Nov. 13, 2008); (8) PCT Patent Application PCT/US2008/014001 (filed on Dec. 23, 2008); (9) U.S. Pat. No. 6,107,102 (issued on Aug. 22, 2000); (10) U.S. Patent Application Publication No. 2008/0311182 (published on Dec. 18, 2008); (11) PCT Patent Application PCT/US2009/000239 (filed on Jan. 15, 2009); (12) PCT Patent Application PCT/US11127746 (filed on Mar. 9, 2011); (13) U.S. Patent Application Publication No. 2010/0029785 (published on Feb. 4, 2010); (14) Tasciotti et al, 2008 Nature Nanotechnology 3:151-157; and (15) PCT Patent Application PCT/US11/28861 (filed on Mar. 17, 2011).

Definitions

Unless otherwise specified “a” or “an” means one or more.

“Nanoporous” or “nanopores” refers to pores with an average size of less than 1 micron.

“Biodegradable” refers to a material that can dissolve or degrade in a physiological medium or a biocompatible polymeric material that can be degraded under physiological conditions by physiological enzymes and/or chemical conditions.

“Biocompatible” refers to a material that, when exposed to living cells, will support an appropriate cellular activity of the cells without causing an undesirable effect in the cells such as a change in a living cycle of the cells; a change in a proliferation rate of the cells or a cytotoxic effect.

“Microparticle” refers to a particle having a maximum dimension from 1 micrometer to 1000 micrometers, or, in some embodiments from 1 micron to 100 microns as specified.

“Nanoparticle” refers to a particle having a maximum dimension of less than 1 micron. The term “theranostic” refers to a delivery system, which may be used to at least one of treating, preventing, monitoring or diagnosing of a physiological condition or a disease.

Introduction

As discussed in more detail below, the present invention provides modified stem cells that comprise a delivery system. The delivery system comprises at least one microparticle or nanoparticle that further comprises an active agent (e.g., a therapeutic agent and/or imaging agent). The present invention also provides pharmaceutical compositions that comprise: (1) the above-described modified stem cell; and (2) a pharmaceutically acceptable carrier. Further embodiments of the present invention pertain to the delivery of the above-described modified stem cells to a subject. Additional embodiments of the present invention pertain to methods of modifying stem cells to make the above-described delivery systems.

In some embodiments, the above-described systems and methods may be used for the treatment, prevention, monitoring and diagnosis of conditions associated with inflammation (e.g., cancer). This combinational platform may provide an ability for the selective and timely release of an active agent, such as a therapeutic and/or imaging agent, at the site of inflammation.

Although the literature shows several approaches that detail using stem cells for the delivery of diagnostic agents, few provide therapeutic relief and home to the target as the present stem cells do (i.e., nearly 100% of the injected dose accumulates at the tumor site in cancer models in some embodiments shown below). Thus, the current invention may provide a mechanism to selectively deliver large payloads of both therapeutic and/or imaging agents or vectors to inflamed tissues upon migration. The use of multistage delivery systems, such as multistage delivery systems utilizing porous silicon particles, may allow for the ability to facilitate the internalization of a formulation, such as a nanoparticle formulation, which may be embedded within the porous matrix of the porous silicon particles, at extremely efficient rates.

The present invention may not only allow the delivery of the multistage delivery systems but also provide the means for delivering other microparticle or nanoparticle-based formulations (not necessarily multistage ones). In various embodiments, such microparticles or nanoparticles may be within stem cells or conjugated to the surface of stem cells. Furthermore, methods that result in the induced release of an active agent (such as a therapeutic agent and/or an imaging agent, and/or microparticles or nanoparticles that may contain an imaging agent) at a target site (such as an inflammation site or a tumor site) are herein described.

To date, the delivery of active agents directly to a site of inflammation may be non-optimal. Furthermore, methods of imaging the site of inflammation may be needed. The inflammation involved in cancer is well established, and is believed to aid in the progression, survival and growth of tumors. Effective methods for the treatment and imaging of an inflammation site, such as a tumor/cancer site, may provide a solution for treating millions of patients. Moreover, the ability to quickly deliver an active agent to sites of injury resulting in the timely repair of the surrounding tissues may be of critical importance for healthcare. Therefore, in some embodiments, the present invention may hold promise to impact the treatment of both military personnel and civilians by providing effective methods for the controlled and site-specific delivery of therapeutics and diagnostics for the treatment of various inflammatory disorders (and other conditions in some embodiments).

Tumors may not only be influenced solely by cytotoxic or mitogenic mechanisms, but also by mechanisms related to inflammation. Currently, cancer is associated with a lifetime risk of 1:2 in men and 1:3 in women. In addition, cancer accounts for nearly 25% of deaths in the United States. The major obstacle facing cancer treatment may be the lack of effective approaches that efficiently deliver active agents, such as therapeutic and/or imaging agents, to the tumor site while sparing normal tissues. The existing treatment and imaging of cancer may have mostly thus far relied heavily on non-targeted agents that have yielded minimal clinical successes, possibly due to the limited concentrations that amass tumors and undesired side effects on normal tissues.

Equally as important to therapy may be the timely detection of tumors, which may be crucial to prevent the progression of advanced cancers and to diagnose cancer relapse post-therapy. Current noninvasive imaging of cancer relies on the use of contrast agents that take advantage of increased metabolic and amino acid metabolism within tumors, but these are limited by background noise and nonspecific uptake. Thus, the development of novel targeted approaches with the potential of delivering therapeutic and diagnostic agents directly to the target site, such as a tumor site and/or an inflammation site, may be needed.

Accordingly, one embodiment of the present invention relates to a modified stem cell(s) comprising a delivery system that comprises at least one microparticle or nanoparticle, which may contain an active agent, such as an imaging agent and/or a therapeutic agent. Such modified stem cells may be used as a part of a composition for treating, monitoring, preventing, staging and/or diagnosing a disease or condition, including a disease or condition associated with inflammation, such as cancer.

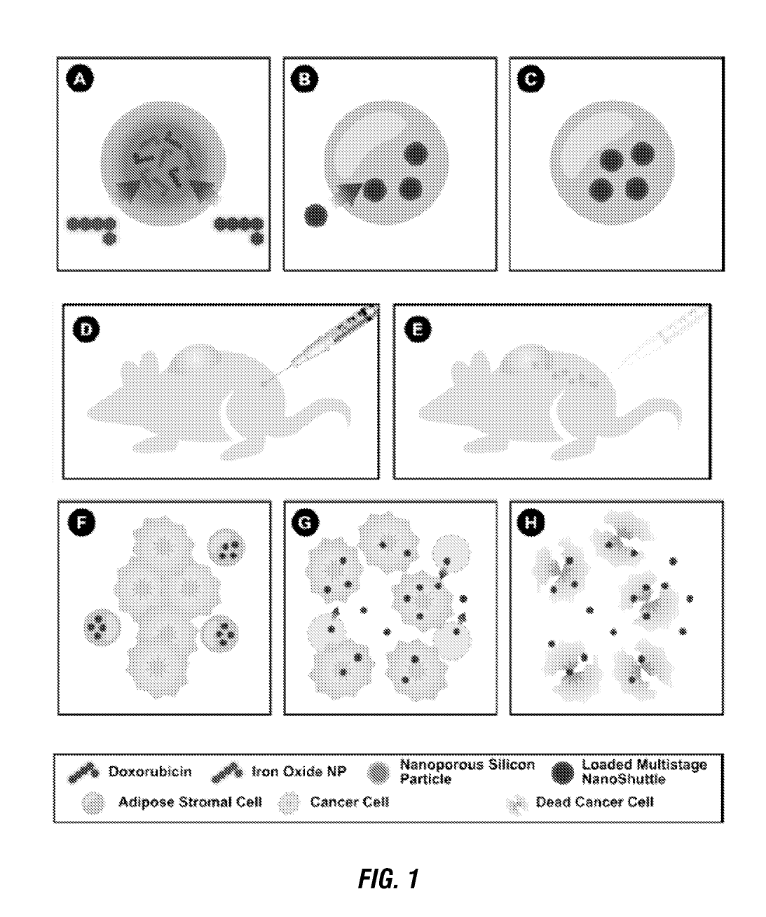

As discussed below and illustrated in FIG. 1, the present invention develops an innovative platform capable of effectively delivering microparticle or nanoparticle based delivery systems for the treatment, monitoring, prevention and diagnosis of various conditions (e.g., cancer and other disorders that induce an inflammatory response). In various embodiments, the targeting may rely on the migration of stem cells (e.g., adipose stem cells or ASCs) to an inflammation site, such as the tumor, which may result in the accumulation of at least 70%, at least 80%, at least 90%, or about 100% of the injected dose of the delivery systems at the inflammation site, such as a tumor site.

In some embodiments, the stem cell may be modified or combined with a multistage delivery system, such as the ones that are disclosed, for example, in U.S. Patent Application Publications Nos. 2008/0311182 and 2008/0280140, as well as in Tasciotti et al, 2008. Nature Nanotechnology. 3:151-157. Without being bound by theory, multistage delivery systems may provide an ability to engineer key physiochemical features, tailor the pharmacological regimen, and control the delivery of the functional payload to the target site. Multistage delivery systems may be loaded, for example, with cytotoxic and imaging NPs, anti-inflammatory drugs, steroids, and proteins capable of providing the optimal therapeutic and diagnostic carrier for diverse pathologies. Moreover, the ability of these stem cells, such as ASCs, to migrate to sites of inflammation allows them to be used as universal carriers for multistage delivery systems for the treatment, imaging or diagnosis of a multitude of pathologies that elicit an inflammatory response. For example, wound healing upon internal and external injuries is associated with the same inflammatory processes governing tumor growth. Therefore, delivery of therapeutic agent, that may aid in tissue healing (rather than toxicity) with delivery systems based on stem cells, such as ASC, may become generally useful in regenerative medicine.

The present invention may also be useful not only for cancer treatment, but also for site-specific, individualized therapy for a multitude of inflammatory diseases. The stem cell delivery platforms of the present invention may substantially impact patient care and provide a strategy that may significantly decrease harsh side effects and avoid unnecessary costs by offering the ability to assess the efficacy early during the course of treatment, allowing for prompt interventions and a switch to an alternative therapeutic strategy.

As described in more detail below, the current invention proposed herein is illustrated in FIG. 1 as a specific and non-limiting embodiment. The present invention decouples the tasks of targeting and therapy onto two distinct components. The stem cells can provide the targeting component, while the microparticles or nanoparticles (hereinafter “particles”) can provide the therapeutic and diagnostic regimen.

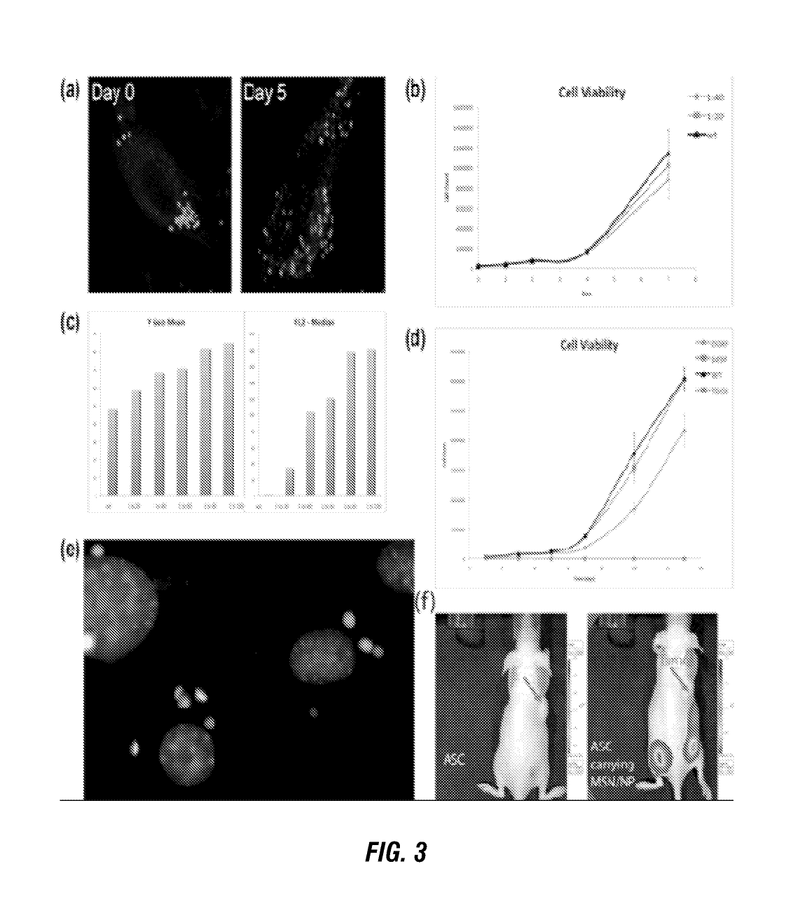

As illustrated in FIGS. 3A-3B, it was established that adipose stromal stem cells (ASCs) were capable of efficiently internalizing a large payload of multistage delivery systems. The results also showed that the multistage delivery systems were biocompatible with ASCs. In addition, the sub-cellular localization of the multistage delivery systems was characterized. It was concluded that the uptake of multistage delivery systems did not induce any significant change in their cytoskeletal structure and overall viability.

Moreover, by formulating doxorubicin (DOX) in micelles and by loading them within the multistage delivery systems, it was possible to achieve the delayed delivery of a cytotoxic therapeutic in a timely manner. This result may be particularly important, as the premature release (or leakage) of the drug from the multistage delivery system would result in the death of the ASC as they migrate to the target. See FIG. 3D.

Lastly, by injecting ASCs loaded with the multistage delivery systems into mice bearing xenograft cancer, it was demonstrated with near infrared (NIR) optical imaging that multistage delivery systems' uptake by ASC did not interfere with their capacity to migrate and home to the tumor mass within four hours. See FIG. 3F.

According to these studies on internalization and intracellular localization, it is envisioned that the multistage delivery systems accumulate in late endosomes. Although this accumulation will not affect the diagnostic function of the system, endosomal trapping may prevent the release of the payload from delivery systems. To overcome this obstacle, several strategies discussed in the Examples may be used to induce the release of the delivery systems, such as multistage delivery systems, from ASCs upon homing to the target site, such as an inflammation or tumor site.

Clinical Relevance

The current invention may provide a multi-component, multi-stage platform for the simultaneous delivery of therapeutic and diagnostic/imaging agents or microparticles or nanoparticles containing such agents. The delivery systems utilizing stem cells may be useful for the treatment of various conditions, such as cancer treatment, and a multitude of other conditions associated with inflammation. The present delivery platform may substantially impact patient care and significantly decrease harsh side effects and avoid unnecessary costs by offering the ability to assess the efficacy early during the course of treatment, and allowing for timelier interventions and a switch to an alternative therapeutic strategy.

Various aspects of the present invention may also exploit the inherent advantages of each component using the multistage delivery systems to protect and distribute active agents (such as therapeutic and/or diagnostic/imaging agents) and stem cells(such as ASCs) to specifically deliver multistage delivery systems to inflamed tissues, thereby enabling site-specific imaging, monitoring, diagnosis, prevention and/or treatment. The intrinsic flexibility of using delivery systems (such as multistage delivery systems) may offer the advantage of selecting a number of active agents, allowing this approach to be easily adapted for several disorders, which may be eventually translated to improvements in health care through individualized and personal therapy/diagnostics.

Furthermore, various aspects of the stem cell based delivery systems of the present invention may decouple the targeting and therapeutic/diagnostic/imaging functions. The homing ability of stem cells, such as ASCs, may result in high percentage of the administered cells reaching the inflamed areas, which may be nearly 100%. Fabricated delivery systems, such as multistage delivery systems, may offer an ability to engineer key physicochemical features within its structure, allowing for the pharmacological regimen to be tailored for controlling the release of payload at the target site. The integration of these components may result in an outcome whose success is greater than the sum of the individual parts, with the end product having the potential to be used as a “universal carrier” for the treatment, monitoring and/or diagnosis of inflammatory diseases.

As described below, in some embodiments, ASCs may be taken from white adipose tissue and derived in large quantities. The ASCs may then be transplanted after minimal ex vivo manipulation. Autologous ASC transplants are currently ongoing in several clinical trials (e.g. Cytori Therapeutics) for the treatment of patients with cardiovascular and wound healing disorders and have proven to be safe and compatible. Furthermore, multistage delivery systems may be derived from porous silicon, which is a proven biocompatible and biodegradable material that is unlikely to elicit any adverse response from the patient. Taken together, the stem cell-directed delivery systems may be used in animals, such as humans, with minimal toxicity. Thus, the stem cell-directed delivery system may be used in both healthy patients for early diagnosis, and in patients currently undergoing treatment.

Advantages

Various aspects of the present invention may be advantageous to current delivery systems through the decoupling of the targeting and therapy components into separate components. By not relying on the decoration of common recognition molecules to target the microparticle or nanoparticle based delivery system (such as a multistage delivery system), but rather allowing them to be carried to the target site (such as the tumor site or an inflammation site) by a stem cell (such as ASC, within the stem cell or by surface conjugation) may lead to the accumulation of an injected dose of the delivery systems at the target site. In some embodiments, the accumulation may be at least 70%, at least 80%, at least 90%, or about 100%.

The active localization of the stem cell-directed delivery system may be advantageous over current approaches, which at best only deliver a tiny fraction of the injected dose to the tumor. Furthermore, the stem cells may be derived in large quantities from a patient and safely used in transplantation applications after minimal ex vivo manipulation. These cells may be recruited by an inflammatory signal enabling their use as universal carriers for a multitude of conditions associated with inflammation. In some embodiments, the ASCs may be a unique set of stromal cells that have been characterized with the ability to migrate to inflammation sites, such as tumor sites. The specific aspects of the present invention will now be described in more detail below as non-limiting examples.

Delivery Systems

The delivery systems of the present invention generally comprise: (1) at least one microparticle or nanoparticle; and (2) an active agent. In some embodiments, the active agent may be encapsulated within a microparticle or nanoparticle. In some embodiments, the active agent may be adhered to or conjugated on a surface of a microparticle or nanoparticle. In further embodiments, active agent is on a surface and inside a microparticle or nanoparticle.

In various embodiments, the particles of the present invention may also have a functionalized surface. For instance, in various embodiments, a surface of a particle may be functionalized with functionalizing agents such as peptides, polymers, chitosans, contrasting agents, imaging agents and calcium phosphates. In more specific embodiments, a surface of a particle may be functionalized with a polymer that becomes swellable in response to a stimulus (e.g., change in temperature, change in pH, change in pressure, and combinations thereof).

Various microparticles and nanoparticles may be used in the delivery systems of the present invention. In various embodiments, the microparticle or nanoparticle is at least one of multistage particles, porous particles, porous silicon particles, porous silica particles, non-porous particles, fabricated particles, polymeric particles, synthetic particles, semiconducting particles, viruses, gold particles, silver particles, quantum dots, indium phosphate particles, iron oxide particles, micelles, lipid particles, liposomes, silica particles, mesoporous silica particles, PLGA-based particles, gelatin-based particles, carbon nanotubes, fullerenes, and combinations thereof.

The microparticles or nanoparticles of the delivery systems of the present invention may also have a variety of shapes and sizes. The dimensions of the microparticles or nanoparticles are not particularly limited and may depend on a particular application. For example, for intravascular administration, a maximum characteristic size of the particle may be smaller than a radius of the smallest capillary in a subject, which is about 4 to 5 microns for humans. In some embodiments, the maximum characteristic size of the particle may be less than about 100 microns, less than about 50 microns, less than about 20 microns, less than about 10 microns, less than about 5 microns, less than about 4 microns, less than about 3 microns, less than about 2 microns, or less than about 1 micron. Yet, in some embodiments, the maximum characteristic size of the particle may be from 100 nm to 3 microns, from 200 nm to 3 microns, from 500 nm to 3 microns, or from 700 nm to 2 microns. In further embodiments, the maximum characteristic size of the particle may be greater than about 2 microns, greater than about 5 microns, or greater than about 10 microns.

Furthermore, the shape of the particles used in the delivery systems of the present invention are not particularly limited. In some embodiments, the particle may be a spherical particle. Yet, in some embodiments, the particle may be a non-spherical particle. In some embodiments, the particle can have a symmetrical shape. Yet, in some embodiments, the particle may have an asymmetrical shape. In some embodiments, the particle may have a selected non-spherical shape configured to facilitate a contact between the particle and a surface of the target site, such as an endothelium surface of the inflamed vasculature. Examples of appropriate shapes include, but are not limited to, an oblate spheroid, a disc, or a cylinder.

In some embodiments, the particle may be such that only a portion of its outer surface defines a shape configured to facilitate a contact between the particle and a surface of the target site (such as an endothelium surface). For example, in some embodiments, the particle can be a truncated oblate spheroidal particle.

The dimensions and shapes of particles of the present invention may be evaluated using methods disclosed in U.S. Patent Application Publications Nos. 2008/0206344 and 2010/0029785. In some embodiments, the microparticles or nanoparticles may be a porous particle, i.e. a particle that comprises a porous material. The porous material may be a porous oxide material or a porous etched material. Examples of porous oxide materials include, but are not limited to, porous silicon oxide, porous aluminum oxide, porous titanium oxide and porous iron oxide. The term “porous etched materials” refers to a material, in which pores are introduced via a wet etching technique, such as electrochemical etching or electroless etching. Examples of porous etched materials include porous semiconductors materials, such as porous silicon, porous germanium, porous GaAs, porous InP, porous SiC, porous SiₓGe₁₋ₓ, porous GaP and porous GaN.

Methods of making porous etched particles are disclosed, for example, U.S. Patent Application Publication No. 2008/0280140. Additional examples of porous particles (such as porous silicon particles and porous silica particles) and methods of making them are disclosed in the following documents: U.S. Pat. Nos. 6,355,270 and 6,107,102; U.S. Patent Publication Nos. 2006/0251562, 2003/0114366 and 2008/0280140; PCT Publication No. WO 2008/021908; Cohen et al., Biomedical Microdevices. 2003. 5(3):253-259; Meade et al., Advanced Materials. 2004. 16(20): 1811-1814; Thomas et al. Lab Chip. 2006. 6:782-787; Meade et al., Phys. Stat. Sol. (RRL) 1(2):R71-R-73 (2007); Salonen et al. Journal of Pharmaceutical Sciences. 2008. 97(2):632-653; Salonen et al., Journal of Controlled Release. 2005. 108:362-374; Foraker, A. B. et al. Pharma. Res. 2003. 20 (1):110-116 (2003); Salonen, J. et al. Jour. Contr. Rel. 2005. 108: 362-374; and Paik J. A. et al. J. Mater. Res. 2003. 17:2121.

In some embodiments, the porous particle may be a nanoporous particle. In some embodiments, an average pore size of the porous particle may be from about 1 nm to about 1 micron, from about 1 nm to about 800 nm, from about 1 nm to about 500 nm, from about 1 nm to about 300 nm, from about 1 nm to about 200 nm, or from about 2 nm to about 100 nm. In some embodiments, the average pore size of the porous particle can be no more than 1 micron, no more than 800 nm, no more than 500 nm, no more than 300 nm, no more than 200 nm, no more than 100 nm, no more than 80 nm, or no more than 50 nm.

In some embodiments, the average pore size of the porous particle can be from about 5 nm to about 100 nm, from about 10 nm to about 60 nm, from about 20 nm to about 40 nm, or from about 10 nm to about 20 nm. In some embodiments, the average pore size of the porous particle can be from about 1 nm to about 10 nm, from about 3 nm to about 10 nm, or from about 3 nm to about 7 nm.

In general, pores sizes may be determined using a number of techniques, including N₂ adsorption/desorption and microscopy, such as scanning electron microscopy (SEM). In some embodiments, pores of the porous particle may be linear pores. Yet, in some embodiments, pores of the porous particle may be sponge like pores.

In some embodiments, at least a portion of the porous particle may comprise a biodegradable region. In many embodiments, the whole particle may be biodegradable. In general, porous silicon may be bioinert, bioactive or biodegradable depending on its porosity and pore size. Also, a rate or speed of biodegradation of porous silicon may depend on its porosity and pore size, see e.g. Canham, Biomedical Applications of Silicon, in Canham LT, editor. Properties of porous silicon. EMIS datareview series No. 18. London: INSPEC. p. 371-376. The biodegradation rate may also depend on surface modification.

In some embodiments, the particle of the present invention may comprise a biodegradable material. For instance, for oral administration, such materials may be a material designed to erode in the GI tract. In some embodiments, the biodegradable particle may be formed of a metal, such as iron, titanium, gold, silver, platinum, copper, and alloys and oxides thereof. In some embodiments, the biodegradable material may be a biodegradable polymer, such as polyorthoesters, polyanhydrides, polyamides, polyalkylcyanoacrylates, polyphosphazenes, and polyesters. Exemplary biodegradable polymers are described, for example, in U.S. Pat. Nos. 4,933,185, 4,888,176, and 5,010,167. More specific examples of such biodegradable polymer materials include poly(lactic acid), polyglycolic acid, polyglycolic-lactice acid (PGLA); polycaprolactone, polyhydroxybutyrate, poly(N-palmitoyl-trans-4-hydroxy-L-proline ester) and poly(DTH carbonate).

A person of ordinary skill in the art will also recognize that the microparticles or nanoparticles of the present invention may be prepared using a number of techniques. In some embodiments, the particles may be produced utilizing a top-down microfabrication or nanofabrication technique, such as photolithography, electron beam lithography, X-ray lithography, deep UV lithography, nanoimprint lithography or dip pen nanolithography. Such fabrication methods may allow for a scaled up production of particles that are uniform or substantially identical in dimensions.

In some embodiments, the particles used in the delivery systems of the present invention may be a multistage particle (also referred to as a multistage delivery system). Such multistage particles generally comprise a larger first stage microparticle or nanoparticle that may contain one or more smaller size second stage particles. Multistage particles are disclosed, for example, in U.S. Patent Application Publication Nos. 2008/0311182 and 2008/0280140. Multistage particles are also disclosed in Tasciotti et al. 2008. Nature Nanotechnology. 3:151-157.

In many embodiments, the first stage particle of the multistage delivery object may already contain one or more second stage particles when the multistage system is introduced in a stem cell, such ASC. For example, when the first stage particle is a porous particle, its pores may be loaded with one or more second stage particles prior to the introduction of the multistage system into the stem cell. After the second stage particles are loaded, the pores of the porous first stage particle may be sealed or capped prior to the introduction of the multistage system into the stem cell.

Various additional particles that may be used as delivery systems in the present invention are disclosed in PCT Publications Nos. WO 2008/041970 and WO 2008/021908; U.S. Patent Application Publications Nos. 2008/0102030, 2003/0114366, 2008/0206344, 2008/0280140, 2010/0029785 and 2008/0311182; PCT Patent Application Nos. PCT/US2008/014001 (filed on Dec. 23, 2008), PCT/US2009/000239 (filed on Jan. 15, 2009), and PCT/US11/27746 (filed on Mar. 9, 2011); and U.S. Pat. No. 6,107,102 and 6,355,270.

A person of ordinary skill in the art will also recognize that various methods of loading active agents into particles may be used. For instance, various methods of loading active agents into porous particles are disclosed, for example, in U.S. Pat. No. 6,107,102 and U.S. Patent Application Publication No. 2008/0311182. In some embodiments, after the active agent is loaded, the pores of the porous particle may sealed or capped prior to introducing the particle into the stem cell.

Active Agents

A person of ordinary skill in the art will also recognize that various active agents may be used in the present invention. In various embodiments, the active agent may be a therapeutic agent, an imaging agent or a combination thereof. In some embodiments, the selection of the active agent may depend on a desired application. Non-limiting examples of active agents are described below.

Therapeutic Agents

A therapeutic agent may be a physiologically or pharmacologically active substance that can produce a desired biological effect in a targeted site in an animal, such as a mammal or a human. The therapeutic agent may be any inorganic or organic compound. Examples include, without limitation, peptides, proteins, nucleic acids (including siRNA, miRNA and DNA), polymers, and small molecules. In various embodiments, the therapeutic agents may be characterized or uncharacterized.

Furthermore, therapeutic agents of the present invention may be in various forms. Such forms may include, without limitation, unchanged molecules, molecular complexes, and pharmacologically acceptable salts (e.g., hydrochloride, hydrobromide, sulfate, laurate, palmitate, phosphate, nitrite, nitrate, borate, acetate, maleate, tartrate, oleate, salicylate, and the like). For acidic therapeutic agent, salts of metals, amines or organic cations (e.g., quaternary ammonium) can be used. Derivatives of drugs, such as bases, esters and amides also can be used as a therapeutic agent.

In some embodiments, a therapeutic agent that is water insoluble can be used in a form that is a water soluble derivative thereof, or as a base derivative thereof. In such instances, the derivative therapeutic agent may be converted to the original therapeutically active form upon delivery to a targeted site. Such conversions can occur by various metabolic processes, including enzymatic cleavage, hydrolysis by the body pH, or by other similar processes.

Non-limiting examples of therapeutic agents include anti-inflammatory agents, anti-cancer agents, anti-proliferative agents, anti-vascularization agents, wound repair agents, tissue repair agents, thermal therapy agents, and combinations thereof.

More specific examples of therapeutic agents include, but are not limited to, anti-cancer agents, such as anti-proliferative agents and anti-vascularization agents; antimalarial agents; OTC drugs, such as antipyretics, anesthetics, cough suppressants; antiinfective agents; antiparasites, such as anti-malaria agents (e.g., Dihydroartemisin); antibiotics, such as penicillins, cephalosporins, macrolids, tetracyclines, aminglycosides, and anti-tuberculosis agents; antifungal/antimycotic agent; genetic molecules, such as anti-sense oligonucleotides, nucleic acids, oligonucleotides, DNA, and RNA; anti-protozoal agents; antiviral agents, such as acyclovir, gancyclovir, ribavirin, anti-HIV agents and anti-hepatitis agents; anti-inflammatory agents, such as NSAIDs, steroidal agents and cannabinoids; anti-allergic agents, such as antihistamines and fexofenadine; bronchodilators; vaccines or immunogenic agents, such as tetanus toxoid, reduced diphtheria toxoid, acellular pertussis vaccine, mums vaccine, smallpox vaccine, anti-HIV vaccines, hepatitis vaccines, pneumonia vaccines and influenza vaccines; anesthetics, including local anesthetics; antipyretics, such as paracetamol, ibuprofen, diclofenac and aspirin; agents for treatment of severe events, such as cardiovascular attacks, seizures and hypoglycemia; anti-nausea and anti-vomiting agents; immunomodulators and immunostimulators; cardiovascular drugs, such as beta-blockers, alpha-blockers and calcium channel blockers; peptide and steroid hormones, such as insulin, insulin derivatives, insulin detemir, insulin monomeric, oxytocin, LHRH, LHRH analogues, adreno-corticotropic hormone, somatropin, leuprolide, calcitonin, parathyroid hormone, estrogens, testosterone, adrenal corticosteroids, megestrol, progesterone, sex hormones, growth hormones and growth factors; peptide and protein related drugs, such as amino acids, peptides, polypeptides and proteins; vitamins, such as Vitamin A, vitamins in the Vitamin B group, folic acid, Vitamin C, Vitamin D, Vitamin E, Vitamin K, niacin, and derivatives of Vitamin D; Autonomic Nervous System Drugs; fertilizing agents; antidepressants, such as buspirone, venlafaxine, benzodiazepins, selective serotonin reuptake inhibitors (SSRIs), sertraline, citalopram, tricyclic antidepressants, paroxetine, trazodone, lithium, bupropion, sertraline, fluoxetine; agents for smoking cessation, such as bupropion, nicotine; lipid-lowering agents, such as inhibitors of 3 hydroxy-3-methylglutaryl-coenzyme A (HMG-CoA) reductase, simvastatin, atrovastatinl; agents for CNS or spinal cord, such as benzodiazepines, lorazepam, hydromorphone, midazolam, Acetaminophen, 4′-hydroxyacetanilide, barbiturates and anesthetics; anti-epileptic agents, such as valproic acid and its derivatives and carbamazepin; angiotensin antagonists, such as valsartan; anti-psychotic agents and anti-schizophrenic agents, such as quetiapine and risperidone; agents for treatment of Parkinsonian syndrome, such as L-dopa and its derivatives and trihexyphenidyl; anti-Alzheimer agents, such as cholinesterase inhibitors, galantamine, rivastigmine, donepezil, tacrine, memantine and N-methyl D-aspartate (NMDA) antagonists; agents for treatment of non-insulin dependent diabetes, such as metformine; agents for treatment of erectile dysfunction, such as sildenafil, tadalafil, papaverine, vardenafil and PGE1; prostaglandins; agents for bladder dysfunction, such as oxybutynin, propantheline bromide, trospium and solifenacin succinate; agents for treatment of menopausal syndromes, such as estrogens and non-estrogen compounds; agents for treatment of hot flashes in postmenopausal women; agents for treatment of primary or secondary hypogonadism, such as testosterone; cytokines, such as TNF, interferons, IFN-α, IFN-β and interleukins; CNS stimulants; muscle relaxants; anti paralytic gas agents; narcotics and antagonists, such as opiates, oxycodone; painkillers, such as opiates, endorphins, tramadol, codein, NSAIDs and gabapentine; Hypnotics, such as zolpidem, benzodiazepins, barbiturates, ramelteon; histamines and antihistamines; anti-migraine drugs, such as imipramine, propranolol and sumatriptan; diagnostic agents, such as phenolsulfonphthalein, Dye T-1824, vital dyes, potassium ferrocyanide, secretin, pentagastrin and cerulein; topical decongestants or anti-inflammatory agents; anti-acne agents, such as retinoic acid derivatives, doxicillin and minocyclin; ADHD related agents, such as methylphenidate, dexmethylphenidate, dextroamphetamine, d- and l-amphetamin racemic mixture and pemoline; diuretic agents; anti-osteoporotic agents, such as. bisphosphonates, aledronate, pamidronate, tirphostins; osteogenic agents; anti-asthma agents; anti-spasmotic agents, such as papaverine; agents for treatment of multiple sclerosis and other neurodegenerative disorders, such as mitoxantrone, glatiramer acetate, interferon β-1α. interferon β-1β; and plant derived agents from leaves, roots, flowers, seeds, stems, branches or extracts.

In various embodiments, the therapeutic agents of the present invention can also be a chemotherapeutic agent, an immunosuppressive agent, a cytokine, a cytotoxic agent, a nucleolytic compound, a radioactive isotope, a receptor, or a pro-drug activating enzyme. The therapeutic agents of the present invention may be naturally occurring or produced by synthetic or recombinant methods, or any combination thereof.

In additional embodiments, therapeutic agents of the present invention may be drugs that are affected by classical multidrug resistance. Non-limiting examples of such drugs include vinca alkaloids (e.g., vinblastine and vincristine), the anthracyclines (e.g., doxorubicin and daunorubicin), RNA transcription inhibitors (e.g., actinomycin-D), and microtubule stabilizing drugs (e.g., paclitaxel).

In more specific and preferred embodiments, the therapeutic agent can be a cancer chemotherapy agent. Non-limiting examples of suitable cancer chemotherapy agents include, without limitation, nitrogen mustards, nitrosorueas, ethyleneimine, alkane sulfonates, tetrazine, platinum compounds, pyrimidine analogs, purine analogs, antimetabolites, folate analogs, anthracyclines, taxanes, vinca alkaloids, topoisomerase inhibitors, and hormonal agents. Exemplary chemotherapy drugs that can be used as chemotherapy agents include, without limitation, Actinomycin-D, Alkeran, Ara-C, Anastrozole, Asparaginase, BiCNU, Bicalutamide, Bleomycin, Busulfan, Capecitabine, Carboplatin, Carboplatinum, Carmustine, CCNU, Chlorambucil, Cisplatin, Cladribine, CPT-11, Cyclophosphamide, Cytarabine, Cytosine arabinoside, Cytoxan, Dacarbazine, Dactinomycin, Daunorubicin, Dexrazoxane, Docetaxel, Doxorubicin, DTIC, Epirubicin, Ethyleneimine, Etoposide, Floxuridine, Fludarabine, Fluorouracil, Flutamide, Fotemustine, Gemcitabine, Herceptin, Hexamethylamine, Hydroxyurea, Idarubicin, Ifosfamide, Irinotecan, Lomustine, Mechlorethamine, Melphalan, Mercaptopurine, Methotrexate, Mitomycin, Mitotane, Mitoxantrone, Oxaliplatin, Paclitaxel, Pamidronate, Pentostatin, Plicamycin, Procarbazine, Rituximab, Steroids, Streptozocin, STI-571, Streptozocin, Tamoxifen, Temozolomide, Teniposide, Tetrazine, Thioguanine, Thiotepa, Tomudex, Topotecan, Treosulphan, Trimetrexate, Vinblastine, Vincristine, Vindesine, Vinorelbine, VP-16, Xeloda, and Camptothecin.

Additional cancer chemotherapy drugs that can be used as therapeutic agents include, without limitation alkylating agents, such as Thiotepa and cyclosphosphamide; alkyl sulfonates such as Busulfan, Improsulfan and Piposulfan; aziridines, such as Benzodopa, Carboquone, Meturedopa and Uredopa; ethylenimines and methylamelamines, including altretamine, triethylenemelamine, trietylenephosphoramide, triethylenethiophosphaoramide and trimethylolomelamine; nitrogen mustards, such as Chlorambucil, Chlornaphazine, Cholophosphamide, Estramustine, Ifosfamide, mechlorethamine, mechlorethamine oxide hydrochloride, Melphalan, Novembiehin, Phenesterine, Prednimustine, Trofosfamide, and uracil mustard; nitroureas, such as Cannustine, Chlorozotocin, Fotemustine, Lomustine, Nimustine, and Ranimustine; antibiotics, such as Aclacinomysins, Actinomycin, Authramycin, Azaserine, Bleomycins, Cactinomycin, Calicheamicin, Carabicin, Carminomycin, Carzinophilin, Chromoinycins, Dactinomycin, Daunorubicin, Detorubicin, 6-diazo-5-oxo-L-norleucine, Doxorubicin, Epirubicin, Esorubicin, Idambicin, Marcellomycin, Mitomycins, mycophenolic acid, Nogalamycin, Olivomycins, Peplomycin, Potfiromycin, Puromycin, Quelamycin, Rodorubicin, Streptonigrin, Streptozocin, Tubercidin, Ubenimex, Zinostatin, and Zorubicin; anti-metabolites, such as Methotrexate and 5-fluorouracil (5-FU); folic acid analogues such as Denopterin, Methotrexate, Pteropterin and Trimetrexate; purine analogs, such as Fludarabine, 6-mercaptopurine, Thiamiprine, and Thioguanine; pyrimidine analogs, such as Ancitabine, Azacitidine, 6-azauridine, Carmofur, Cytarabine, Dideoxyuridine, Doxifluridine, Enocitabine, Floxuridine, and 5-FU; androgens, such as Calusterone, Dromostanolone Propionate, Epitiostanol, Rnepitiostane and Testolactone; anti-adrenals, such as aminoglutethimide, Mitotane and Trilostane; folic acid replenisher, such as frolinic acid; aceglatone; aldophosphamide glycoside; aminolevulinic acid; Amsacrine; Bestrabucil; Bisantrene; Edatraxate; Defofamine; Demecolcine; Diaziquone; Elfornithine; elliptinium acetate; Etoglucid; gallium nitrate; hydroxyurea; Lentinan; Lonidamine; Mitoguazone; Mitoxantrone; Mopidamol; Nitracrine; Pentostatin; Phenamet; Pirarubicin; podophyllinic acid; 2-ethylhydrazide; Procarbazine; PSK®; Razoxane; Sizofrran; Spirogermanium; tenuazonic acid; triaziquone; 2, 2′,2″-trichlorotriethylamine; Urethan; Vindesine; Dacarbazine; Mannomustine; Mitobronitol; Mitolactol; Pipobroman; Gacytosine; Arabinoside (“Ara-C”); cyclophosphamide; thiotEPa; taxoids, such as Paclitaxel (TAXOL®, Bristol-Myers Squibb Oncology, Princeton, N.J.) and Doxetaxel (TAXOTERE®, Rhone-Poulenc Rorer, Antony, France); Chlorambucil; Gemcitabine; 6-thioguanine; Mercaptopurine; Methotrexate; platinum analogs, such as Cisplatin and Carboplatin; Vinblastine; platinum; etoposide (VP-16); Ifosfamide; Mitomycin C; Mitoxantrone; Vincristine; Vinorelbine; Navelbine; Novantrone; Teniposide; Daunomycin; Aminopterin; Xeloda; Ibandronate; CPT-11; topoisomerase inhibitor RFS 2000; difluoromethylomithine (DMFO); retinoic acid; Esperamicins; Capecitabine; and pharmaceutically acceptable salts, acids or derivatives of any of the above.

In additional embodiments, therapeutic agents of the present may also include anti-hormonal agents that act to regulate or inhibit hormone action on tumors, such as anti-estrogens (e.g., without limitation Tamoxifen, Raloxifene, aromatase inhibiting 4(5)-imidazoles, 4 Hydroxytamoxifen, Trioxifene, Keoxifene, Onapristone, and Toremifene (Fareston)); anti-androgens (e.g., without limitation, Flutamide, Nilutamide, Bicalutamide, Leuprolide, and Goserelin); and pharmaceutically acceptable salts, acids or derivatives of any of the above.

Cytokines can be also used as the therapeutic agents in various embodiments of the present invention. Examples of such cytokines are lymphokines, monokines, and traditional polypeptide hormones. Included among the cytokines are also growth hormones, such as human growth hormone, N-methionyl human growth hormone, and bovine growth hormone; parathyroid hormone; thyroxine; insulin; proinsulin; relaxin; prorelaxin; glycoprotein hormones, such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors, such as NGF-β; platelet growth factor; transforming growth factors (TGFs), such as TGF-α and TGF-β; insulin-like growth factor-I and -II; erythropoietin (EPO); osteoinductive factors; interferons, such as interferon-a, -β and -γ; colony stimulating factors (CSFs), such as macrophage-CSF (M-CSF), granulocyte-macrophage-CSF (GM-CSF), and granulocyte-CSF (GCSF); interleukins (ILs), such as IL-1, IL-1α, IL-2, IL-3, IL-4, IL-5, IL-6, IL-7, IL-8, IL-9, IL-11, IL-12, and IL-15; tumor necrosis factors, such as TNF-α or TNF-β; and other polypeptide factors, including LIF and kit ligand (KL). As used herein, the term cytokine includes proteins from natural sources, or from recombinant cell culture and biologically active equivalents of the native sequence cytokines. In some embodiments, the therapeutic agent can be an antibody-based therapeutic agent, such as Herceptin, Erbitux, Avastin, Rituxan, Panitumumab, Mylotarg, Zenapax, Simulect, Enbrel, Adalimumab, and Remicade.

In some embodiments, the therapeutic agent can be a nanoparticle. For example, in some embodiments, the nanoparticle can be a nanoparticle that can be used for a thermal ablation or a thermal therapy. Examples of such nanoparticles include any metal and semiconductor based nanoparticle, which includes but is not limited to: iron oxide, quantum dots (both CdSe and indium phosphate), gold (spherical, rods, hollow nanoshperes), silver, carbon nanotubes, carbon fullerenes, silica, and silicon nanoparticles.

Imaging agent

Imaging agents in the present invention may be substances that provide imaging information about a targeted site in a body of an animal, such as a mammal or a human being. In some embodiments, the imaging agent may comprise a magnetic material, such as iron oxide or a gadolinium containing compound. In additional embodiments, such imaging agents may be utilized for magnetic resonance imaging (MRI).

For embodiments involving optical imaging, the imaging agent may be, for example, semiconductor nanocrystals or quantum dots. For optical coherence tomography imaging, the imaging agent may be a metal, such as gold or silver nanocage particles. In some embodiments, the imaging agent may be metal nanoparticles, such as gold or silver nanoparticles. In additional embodiments, the imaging agents may be semiconductor nanoparticles, such as quantum dots.

In some embodiments, the imaging agent may be an ultrasound contrast agent, such as a microbubble, a nanobubble, an iron oxide microparticle, or an iron oxide nanoparticle. In some embodiments, the imaging agent may be a molecular imaging agent that can be covalently or non-covalently attached to a particle's surface.

In some embodiments, the imaging agent may be a metal ion complex/conjugate that can be covalently or non-covalently attached to a particle's surface. In some embodiments, the imaging agent may be a radionucleotide that can be covalently or non-covalently attached to a particle's surface.

As described above, various particles and active agents may be used in the delivery systems of the present invention. As set forth below, various methods may also be used to load the above-mentioned delivery systems into various stem cells.

Stem Cell Modification

Various aspects of the present invention provide methods of modifying a stem cells by associating a delivery system of the present invention (e.g., a microparticle or nanoparticle associated with at least one active agent) with the stem cell. In some embodiments, the association can occur by introducing (i.e., “loading”) a delivery system inside the stem cells. In additional embodiments, the association can occur by conjugating or adhering a delivery system to a surface of stem cells. In some embodiments, the conjugation or adhesion may be facilitated by one or more functional groups on a surface of a delivery system.

Accordingly, in some embodiments, the delivery system is on a surface of the modified stem cell. In some embodiments, the delivery system is inside the modified stem cell. In some embodiments, the delivery system is inside and on a surface of the modified stem cell.

A person of ordinary skill in the art will also recognize that delivery systems of the present invention may be introduced (i.e., “loaded”) into or onto various stem cells (e.g., ASCs) by a number of methods. In some embodiments, loading may involve incubating a microparticle or nanoparticle with stem cells. In such embodiments, the stem cells may passively take up the particles of the delivery system through endocytosis when being incubated together. To facilitate the endocytosis, particles of the delivery system may also be selected to have a specific size and/or shape. Methods of selecting a particle's sizes and/or shapes to facilitate the endocytosis are disclosed, for example, in US Patent Application Publication Nos. 2008/0206344 and 2010/0029785.

In some embodiments, a surface of the microparticle or nanoparticle based delivery system may be modified or functionalized with a functionalizing agent in order to improve the internalization by the stem cell. Non-limiting examples of suitable functionalizing agents include peptides, polymers, coatings (e.g., chitosans and/or calcium phosphates) and the like.

For example, in some embodiments, a surface of the microparticle or nanoparticle based delivery system may be modified or functionalized with a biological molecule, such as a peptide, to improve the internalization by the stem cell through peptide mediated internalization. One non-limiting example of appropriate biological molecule may be nuclear localization signals (e.g., HIV-1 Tat basic peptide).

In some embodiments, a surface of the microparticle or nanoparticle based delivery system may be modified or functionalized with pH sensitive polymeric swelling agents to improve the internalization by the stem cell. In some embodiments, a surface of the micro or nanoparticle based delivery system may be modified or functionalized with a coating, which may be, for example, a chitosan or a calcium phosphate coating, to improve the internalization by the stem cell.

In some embodiments, a surface of the microparticle or nanoparticle based delivery system may be modified, functionalized or conjugated with appropriate functional groups (such as maleimide head groups) to react and form a stable surface-conjugation on the stem cells using appropriate reactive groups (such as reduced thiol groups). This allows the surface to be conjugated with the delivery systems of the present invention in some embodiments.

The particles of the delivery systems of the present invention may be introduced into various stem cells. Non-limiting examples are set forth below.

Stem Cells

By way of background, stem cells are an effective delivery vehicle for various active agents, including anti-neoplastic medicines. Other sections of this application particularly describe the use of ASCs. The description below includes a discussion of other stem cells viable for use in the current invention.

Examples of stem cells suitable for use with various embodiments of the present invention include, without limitation adult stem cells, embryonic stem cells, fetal stem cells, mesenchymal stem cells, neural stem cells, totipotent stem cells, pluripotent stem cells, multipotent stem cells, oligopotent stem cells, unipotent stem cells, adipose-derived stem cells, and endothelial stem cells. Other suitable stem cells can also be envisioned by persons of ordinary skill in the art.

While not wishing to be bound or limited by theory, the directed migration abilities of stem cells provide an important component for using stem cells to deliver active agents (e.g., anti-neoplastic drugs) to a desired site in an organism (e.g., an inflammation site, such as a cancerous site). For instance, many types of stem cells show a strong tropism toward gliomas, including, but not limited to, neural stem cells, bone marrow mesenchymal stem cells, and undifferentiated embryonic stem cells. See, e.g., Li et al. Neuroreport. 2007. 18(17): 1821-1825; and Aboody et al., Proc Nat Acad Sci USA. 2000. 97(23):12846-12851.

Stem cells can also be derived from various sources. In some embodiments, stem cells may be obtained from a source tissue. Accordingly, whether a stem cell population is derived from adult or embryonic sources, the stem cells can be grown in a culture medium to increase the population of a heterogeneous mixture of cells, or a purified cell population. Several methods of growing stem cells outside of the body have been developed and are known in the art.

The stem cells to be expanded can be isolated from any organ of any mammalian organism, by any means known to one of skill in the art. The stem cells can be derived from embryonic or adult tissue. In some embodiments, the stem cells are mesenchymal stem cells. One of skill of the art can determine how to isolate the stem cells from the particular organ or tissue of interest, using methods known in the art. In one embodiment, the stem cells are isolated from umbilical cord blood. In one embodiment, the stem cells are isolated from bone marrow.

In more specific embodiments, stem cells may be derived or isolated directly from a subject that will be treated with a modified version of the stem cell (i.e., a modified stem cell derived from the subject). In some embodiments, the subject may be a human being suffering from a condition associated with inflammation (e.g., cancer).

A person of ordinary skill in the art will also be able to determine a suitable growth medium for initial preparation of stem cells. Commonly used growth media for stem cells include, but are not limited to, Iscove's modified Dulbecco's Media (IMDM), DMEM, KO-DMEM, DMEM/F12, RPMI 1640 medium, McCoy's 5A medium, minimum essential alpha medium (α-MEM), F-12K nutrient mixture medium (Kaighn's modification, F-12K), X-vivo 20, Stemline, CC100, H2000, Stemspan, MCDB 131 Medium, Basal Media Eagle (BME), Glasgow Minimum Essential Media, Modified Eagle Medium (MEM), Opti-MEM I Reduced Serum Media, Waymouth's MB 752/1 Media, Williams Media E, Medium NCTC-109, neuroplasma medium, BGJb Medium, Brinster's BMOC-3 Medium, CMRL Medium, CO₂-Independent Medium, Leibovitz's L-15 Media, and the like.

If desired, other components, such as growth factors, can be added to the above-mentioned media. Exemplary growth factors and other components that can be added include, but are not limited to, thrombopoietin (TPO), stem cell factor (SCF), IL-1, IL-3, IL-7, flt-3 ligand (flt-3L), G-CSF, GM-CSF, Epo, FGF-1, FGF-2, FGF-4, FGF-20, IGF, EGF, NGF, LIF, PDGF, bone morphogenic proteins (BMP), activin-A, VEGF, forskolin, glucocorticoids, and the like. Furthermore, the media can contain serum from various sources, such as fetal, calf, horse, human, or serum substitution components. Numerous agents can also be introduced into media to alleviate the need for serum. For example, serum substitutes such as bovine serum albumin (BSA), insulin, 2-mercaptoethanol and transferrin (TF) may be used in the above-mentioned media.

The stem cells can then be stored for a desired period of time, if needed. Stem cell storage methods are known to those of skill in the art. For instance, the stem cells may be treated to a cryoprotection process, then stored frozen until needed. Cryoprotective agents are well known to one skilled in the art and can include, without limitation, dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, or choline chloride. The above-described and additional cryoprotective agents are disclosed in U.S. Pat. No. 6,461,645 and incorporated herein by reference.

The stem cells can be purified prior to contact with a controlled-release vehicle by methods known in the art. For instance, antibody technology, such as panning of cells through the use of fluorescence activated cell sorting (FACS) methods may be used for purification. In other embodiments, magnet activated cell sorting (MACS) methods may be used purify stem cells. Such methods may be used to isolate cells having desired stem cell markers. Such methods may also be used to remove unwanted or contaminating cell types having unwanted cell markers. Other methods of stem cell purification or concentration can include the use of techniques such as counterflow centrifugal elutriation, equilibrium density centrifugation, velocity sedimentation at unit gravity, immune resetting, immune adherence, and T-lymphocyte depletion.

Examples of stem cell markers that can be useful in purification include, but are not limited to, FLK-1, AC133, CD34, c-kit, CXCR-4, Oct-4, Rex-1, CD9, CD13, CD29, CD44, CD166, CD90, CD105, SH-3, SH-4, TRA-1-60, TRA-1-81, SSEA-4, Sox-2, and the like. Examples of cell surface markers that can be used as markers of contaminating, unwanted cell types depends on the stem cell phenotype sought. For example, if collection of pluripotent hematopoietic cells is desired, contaminating cells will possess markers of commitment to the differentiated hematopoietic cells, such as CD38 or CD33 Likewise, if selection of stromal mesenchymal cells is desired, then contaminating cells would be detected by expression of hematopoietic markers, such as CD45. Additionally, stem cells can be purified based on properties such as size, density, adherence to certain substrates, or ability to efflux certain dyes (e.g., Hoechst 33342 or Rhodamine 123).

In some embodiments, the stem cells to be modified are human mesenchymal stem cells (MSC). Mesenchymal stem cells are the formative pluripotent blast cells found in the bone marrow and peripheral blood. Mesenchymal stem cells are also commonly referred to as “marrow stromal cells” or just “stromal cells”. MSCs can migrate toward glioma cells because of an inherent specific affinity for glioma cells. See, e.g., Yuan et al. 2006. Cancer Res. 66:2630-2638; and Nakamizo et al. 2005. Cancer Res. 65:3307-3318.

Although MSCs are rare (comprising about 0.01-0.0001% of the total nucleated cells of bone marrow), the cells may be isolated from bone marrow, purified from other bone marrow cells, and expanded in culture without loss of their stem cell potential. See, e.g., Haynesworth S E et al. 1992. Bone. 13:81-88. In some embodiments, the MSC for use in the compositions and methods described herein can be isolated from peripheral blood or bone marrow. A method for preparing MSC has been described in U.S. Pat. No. 5,486,359, Furthermore, mesenchymal stem cells may also be isolated from umbilical cord blood, as described by Erices et al. 2000. Br. J Haematol. 109(1):235-42.

In some embodiments, the MSCs may be isolated from the bone marrow or peripheral blood of a subject, such as a subject afflicted with a glioma who will be the recipient of the treatment (i.e., MSCs may be used in autologous transplantation).

Several suitable techniques that may be used in various embodiments of the present invention for the rapid isolation of mesenchymal stem cells include, but are not limited to, leucopheresis, density gradient fractionation, immunoselection, differential adhesion separation, and the like. For example, immunoselection can include isolation of a population of MSCs using monoclonal antibodies raised against surface antigens expressed by bone marrow-derived MSCs (i.e., SH2, SH3 or SH4), as described, for example, in U.S. Pat. No. 6,387,367. The SH2 antibody binds to endoglin (CD105), while SH3 and SH4 bind to CD73. Further, these monoclonal antibodies provide effective probes which can be utilized for identifying, quantifying and purifying MSC, regardless of their source in the body. In one embodiment, MSCs are culture expanded to enrich for cells expressing CD45, CD73, CD105, stro-1, or a combination thereof. In another embodiment, human MSCs are culture-expanded to enrich for cells containing surface antigens identified by monoclonal antibodies SH2, SH3 or SH4, prior to administering the human MSCs to the subject. A stro-1 antibody is described in Gronthos et al. 1996. J. Hematother. 5:15-23. Further cell surface markers that may be used to enrich for human MSCs are those found in Table I, page 237 of Fibbe et al. 2003. Ann. N.Y. Acad. Sci. 996:235-244.

The stem cells (e.g., MSCs) for use in the compositions, systems and methods of the present invention described herein can be maintained in culture media. Such media can be chemically defined as serum free media. In other embodiments, such media can be a “complete medium”, such as Dulbecco's Modified Eagles Medium supplemented with 10% serum (DMEM). Non-limiting examples of chemically defined serum free media are described in U.S. Pat. No. 5,908,782 and WO96/39487. Non-limiting examples of complete media are described in U.S. Pat. No. 5,486,359. Chemically defined medium comprises a minimum essential medium such as Iscove's Modified Dulbecco's Medium (IMDM) supplemented with human serum albumin, human Ex Cyte lipoprotein, transferrin, insulin, vitamins, essential and non-essential amino acids, sodium pyruvate, glutamine and a mitogen. These media stimulate MSC growth without differentiation. Culture for about 2 weeks results in 10 to 14 doublings of the population of adherent cells.

After plating the cells, removal of non-adherent cells by changes of medium every 3 to 4 days results in a highly purified culture of adherent cells that have retained their stem cell characteristics. Such isolated cells can be identified and quantified by their expression of cell surface antigens by the use of monoclonal antibodies that may be specific for SH2, SH3 and/or SH4.

In some embodiments, the stem cells are neural stem cells (NSC). NSCs can be isolated from post-natal and adult tissues. NSCs derived from post-natal and adult tissues are quantitatively equivalent with respect to their capacity to differentiate into neurons and glia, as well as in their growth and differentiation characteristics. However, the efficiency of in vitro isolation of NSCs from various post-natal and adult CNS can be much lower than isolation of NSCs from fetal tissues which harbor a more abundant population of NSCs.

The NSCs can be derived from one site and transplanted to another site within the same subject as an autograft. Furthermore, the NSCs can be derived from a genetically identical donor and transplanted as an isograft. Still further, the NSCs can be derived from a genetically non-identical member of the same species and transplanted as an allograft. Alternatively, NSCs can be derived from non-human origin and transplanted as a xenograft. With the use of immunosuppressants, allograft and xenograft of non-human neural precursors, such as neural precursors of porcine origin, can be grafted into human subjects.

A sample tissue can be dissociated by any standard method. In an embodiment, tissue is dissociated by gentle mechanical trituration using a pipet and a divalent cation-free saline buffer to form a suspension of dissociated cells. Sufficient dissociation to obtain largely single cells is desired to avoid excessive local cell density.

In another embodiment, a neural stem cell line can be induced to be further enriched for a particular subtype of neurons. A number of growth factors, chemicals, and natural substances have been screened to identify effective inducers of particular neurons such as tyrosine hydroxylase-expressing dopaminergic neurons and acetylcholine-producing cholinergic neurons from NSCs of midbrain or spinal cord. The factor or chemical or combination thereof can be introduced during the mitotic phase and/or the differentiation phase of the NSCs.

Modes of Administration

A person of ordinary skill in the art will also recognize that the stem cell based delivery systems of the present invention may be administered to a subject (e.g., a mammal, such as a human) by various modes in order to treat, prevent, diagnose, and/or monitor a physiological condition (e.g., a disease, such as a form of cancer).

The particular administration method employed for a specific application may be determined by the attending physician. Typically, the delivery systems of the present invention may be administered by one of the following routes: topical, parenteral, inhalation/pulmonary, oral, intraocular, intranasal, bucal, vaginal and anal. The stem cell based delivery systems of the present invention may be particularly useful for oncological applications, i.e. for treatment and/or monitoring cancer or a condition, such as tumor associated with cancer.

The majority of therapeutic applications may involve some type of parenteral administration, which may include intravenous (i.v.), intramuscular (i.m.) and/or subcutaneous (s.c.) injections. Furthermore, administration of the delivery systems of the present invention may be systemic or local.

The non-parenteral examples of administration recited above are examples of local administration. Intravascular administration can be local or systemic. Local intravascular delivery can be used to bring a therapeutic substance to the vicinity of a known lesion by use of a guided catheter system (such as a CAT-scan guided catheter) via portal vein injection. General injection, such as a bolus i.v. injection or continuous/trickle-feed i.v. infusion are typically systemic.

In some embodiments, the delivery systems of the present invention may be administered via i.v. infusion, via intraductal administration or via an intratumoral route. In addition, the delivery systems of the present invention may be formulated in any suitable form.

In some embodiments, the modified stem cells may be administered to a subject, such as a human. In more specific embodiments, the modified stem cells may be administered to a human being suffering from a condition associated with inflammation, such as cancer. In further embodiments the modified stem cells may migrate to a site associated with the condition (i.e., inflammation or cancer) within the subject after administration. Thereafter, the active agent may be released from the modified stem cell after migration to the site.

The active agent may then be released from modified stem cells by various mechanisms. For instance, in some embodiments, the release of the active agent from the modified stem cells may involve lysis of cells. In various embodiments, such lysis of cells may be induced by a stimulus, such as radiofrequency signals, heat, magnetic field radiation, light, changes in pressure, changes in pH, changes in temperature and combinations thereof. In additional embodiments, the release of the active agent from the modified stem cell may comprise time-dependent necrosis of the modified stem cells. Other release mechanisms well known to persons of ordinary skill in the art can also be envisioned.

Thus, in additional embodiments of the present invention, the delivery methods may further comprise a step of inducing the lysis of the modified stem cells by exposing the stem cells to a stimulus. Such a stimulus can be radiofrequency signals, heat, magnetic field radiation, light, changes in pressure, changes in pH, changes in temperature and combinations thereof.

Applications

As set forth previously, the stem cell based delivery systems of the present invention may be used as systems for delivering an active agent, such as a therapeutic and/or imaging agent, to an animal. In many embodiments, the animal may be a warm blooded animal, such as a bird or a mammal. In certain embodiments, the animal may be a human being.

The stem cell based delivery systems of the present invention may also be used for treating, diagnosing, preventing and/or monitoring a number of diseases and conditions. In some embodiments, the stem cell based delivery system may be used for delivering an active agent (such as a therapeutic and/or an imaging agent) to a site affected with cancer (such as a tumor site). Accordingly, in some embodiments, the delivery systems of the present invention may be used to treat, monitor, prevent and/or diagnose various cancers and cancerous conditions, including but not limited to lymphoma, colon cancer, lung cancer, pancreatic cancer, ovarian cancer, breast cancer and brain cancer.

In some embodiments, the stem cell based delivery systems of the present invention may be used to target an inflamed site in a subject, such as an animal. Therefore, in such embodiments, the stem cell based delivery systems of the present invention may be used to treating, prevent, monitor, and/or diagnose a condition or disease associated with an inflammation. Examples of such conditions include, but are not limited to, allergies, asthma, Alzheimer's disease, diabetes, hormonal imbalances, autoimmune diseases (such as rheumatoid arthritis and psoriasis), osteoarthritis, osteoporosis, atherosclerosis (including coronary artery disease), vasculitis, chronic inflammatory conditions (such as obesity and ulcers, including Marjolin's ulcer), respiratory inflammations caused by asbestos or cigarette smoke, foreskin inflammations, inflammations caused by viruses (such as Human papilloma virus, Hepatitis B, Hepatitis C or Ebstein-Barr virus), Schistosomiasis, pelvic inflammatory disease, ovarian epitheal inflammation, Barrett's metaplasia, H. pylori gastritis, chronic pancreatitis, Chinese liver fluke infestation, chronic cholecystitis and inflammatory bowel disease, and inflammation-associated cancers. Non-limiting examples of inflammation-associated cancers include prostate cancer, colon cancer, breast cancer, gastrointestinal tract cancers (such as gastric cancer, hepatocellular carcinoma, colorectal cancer, pancreatic cancer, gastric cancer, nasopharyngeal cancer, esophageal cancer, cholangiocarcinoma, gall bladder cancer and anogenital cancer), intergumentary cancer (such as skin carcinoma); respiratory tract cancers (such as bronchial cancer and mesothelioma); genitourinary tract cancer (such as phimosis, penile carcinoma and bladder cancer); and reproductive system cancer (such as ovarian cancer).

Additional conditions and diseases associated with an inflammation that may be treated with the stem cell based delivery systems of the present invention are disclosed in the following references: (1) M. Macarthur et al. 2004. Am. J. Physiol Gastrointest Livel Physiol. 286:G515-520; (2) Calogero et al. 2007. Breast Cancer Research. v. 9(4); Wienberg et al. 2003. J. Clin. Invest, 112: 1796-1808; and Xu et. al. 2003. J. Clin Invest, 112:1821-1830.

The stem cell based delivery systems of the present invention may also be used to treat tissue repair rather than inducing cancer cell death through their intrinsic ability to home to sites of inflammation. For instance, other investigators have shown the ability of ASCs to target wounds and sites of tissue injury and undergo differentiation. See, e.g., Gimble et al. 2007. Circ Res. 100(9):1249-1260. However, Applicants contend that one can expedite this process by delivering growth factors to quickly repair the damaged tissue and recruit critical nutrients and components to the site of injury.

The inflammation involved in the formation of atherosclerosis is well established. See Libby et al, 2009. J Am Coll Cardiol. 54(23):2129-2138. In this scenario, Applicants potentially could use these stem cell based delivery vehicles (e.g., ASC based vehicles) to home to these plaques and use nanoparticles suitable for imaging their size and location. Once proven successful, Applicants can provide delivery of agents that will aid in the elimination of these plaques. Furthermore, Applicants can optimize treatments of RF that potentially can provide a thermal approach to heat specifically these plaques while reducing adverse side effects.

Additional aspects of the present invention will now be described in more detail below with reference to non-specific examples.

### EXAMPLES

The fabrication of porous nanoparticles and MSNs is known. See, for example, U.S. patent application Ser. No. 11/836,004, 12/110515, and PCT Applications PCT/US2008/061775, PCT/US2008/014001, and PCT/US2009/000239, all incorporated herein by reference. The development of novel targeted approaches with the potential of delivering therapeutic and diagnostic agents directly to the tumor site is urgently needed [3]. Nanoparticles (NP) have emerged as promising platforms capable of delivering cytotoxic and imaging agents to tumor sites at efficacious doses, all the while minimizing adverse side effects [4, 5]. However due to their shape, surface charge, and inadvertent environmental activation, they elicit sequestration by several biological barriers [6, 7]. Advances in engineering the size and shape of NP aided their ability to exploit the enhanced permeability and retention (EPR) effect, allowing NP to passively localize in tumors [8]. However, even when decorated with targeting moieties, NP often fail to accumulate at tumor sites at therapeutically relevant dosages. Moreover, the inherent toxicities of NP when exposed to normal tissues have been claimed as limits or impediments to their clinical success. For this reason, only a handful of NP-based therapeutics has experienced success in the clinic [9].

In order for NP to evolve as effective solutions for systemic administration, they must sequentially evade several biological barriers, all the while retaining their selectivity to reach the tumor site. Given the enormous complexity and the sequential and synergistic mode of action of biological barriers, it is reasonable to believe that bestowing a single NP with all the necessary tools to achieve its intended goal is difficult [6]. However, Using advanced mathematical modeling [10-19] of the microvasculature and blood flow dynamics, it is possible to engineer and test a novel class of particles designed to carry, protect and deliver NP. The particles were designed to decouple the multitude of tasks that are required for a single agent and distribute them to multiple stages [2]. Due to its biodegradability [20], biocompatibility [21], physicochemical properties and the ability to control their size, shape, porosity and pore size

, nanoporous silicon was chosen as the first-stage of this multistage delivery system. We have designed multi stage nanoshuttles (MSN) optimized to carry cytotoxic and imaging NP embedded in their mesoporous matrix, and release them in a controlled fashion [23, 24]. These MSN can be loaded with cytotoxic and imaging NP [2, 25-27], anti-inflammatory drugs [28], steroids [29], and proteins [30, 31], each of which can be tailored to provide the optimal therapeutic and diagnostic features necessary for a diversity of pathologies and applications. Furthermore, the release kinetics of the NP can be linked to the degradation of the MSN by simply adjusting their pore size, porosity or pore distribution thus providing an additional level of control over the system [32]. Finally, the intrinsic versatility of this platform allows for the facile adjustment of multiple payloads, allowing for a change of therapy in the instance of chemoresistance and for the effective treatment of both the tumor and its associated microenvironment. To overcome current limitations in the selective accumulation at its intended location upon systemic administration, novel strategies are urgently needed to direct MSN to the tumor tissue and to ensure their targeted action.

Solid tumors grown beyond a certain size are known to establish a site of chronic inflammation [33]. Mesenchymal stromal cells (MSC), a population of multipotent progenitor cells residing in the stroma of a number of adult organs [34-36], have been shown to sense and respond to inflammatory signals released by tumors by migrating and eventually homing to the cancer mass [37, 38]. A number of studies in experimental animal models have reproducibly concluded that MSC can be extremely efficient as migratory vehicles delivering drugs or gene therapy vectors to tumors [39-41]. Recently, the use of bone marrow-derived MSC (BM-MSC) for in vivo delivery of NP to mouse tumor xenografts using magnetic resonance imaging has been reported [42]. Other groups have described an alternate route for incorporation of payloads on cells by taking advantage of the high levels of reduced thiol groups on their surface and surface-conjugating the payload to ensure its delivery. See, .e.g., Sahaf et al. 2003. Proceedings of the National Academy of Sciences of the United States of America. 100(7): 4001-4005; and Stephan et al. 2010. Nature medicine 16(9): 1035-1041.

A multipotent population of cells from white adipose tissue (WAT) provide an alternative source of MSC and ASCs. A key advantage of ASC over BM-MSC is that they are highly abundant in WAT and can be derived in large quantities through a standard liposuction procedure and used for autologous transplantations with minimal ex vivo manipulation [43-45]. Autologous ASC transplants are currently ongoing in several clinical trials (e.g. Cytori Therapeutics: www.clinicaltrial.gov/ct2/show/ NCT00913289) for the treatment of patients with liver failure, cardiovascular and wound healing disorders and have proven to be safe and compatible. Studies in mouse tumor models demonstrate that, like BM-MSC, transplanted ASC systemically home to experimental tumors [1]. The proof of principle for this approach has been demonstrated using ASC as vectors for the delivery of a suicide gene to tumors [39].

By combining the ability of MSN to protect, load, carry and distribute NP with the selective homing of ASC to inflamed tissue, one embodiment of the current invention shows that ASC can act as a specific vehicle for the targeted delivery of MSN. This approach is illustrated in FIG. 1. Results of patient-derived ASC biodistribution will provide critical information on the optimal ASC administration routes, possible sites of non-specific accumulation, and half-life of transplanted ASC in humans.

One embodiment of the current invention exploits the inherent advantages of MSN for carrying both therapeutic and diagnostic NP and of ASC to specifically deliver loaded MSN to tumors, enabling site-specific imaging and treatment.

Data

Studies have shown the abilities of the individual components of the system: the ASC and the MSN. The ability of mouse WAT-derived ASC to home to tumor sites upon systemic administration (intravenous (i.v.) or subcutaneous (s.c.)) within 1 hour is provided [1]. One week after administration, only a small number of ASC were found in the lungs and none were detected in other control organs (e.g., spleen, pancreas, liver and WAT), resulting in nearly 100% of the injected cells arriving to the tumor site. Ongoing studies as well as published data have demonstrated the ability of MSN to uptake and release NP of different size, composition and structure, thereby validating the potential of MSN to provide therapy and imaging through multiple NP formulations [2, 25-27]. In particular, the ability to reproducibly deliver (through the use of 1×10⁸ MSN) more than 10 μg of micelle-formulated doxorubicin (DOX) is provided. This data is consistent with the reports in the literature that demonstrate that conventional DOX treatments at best can provide the accumulation of 0.8-5 μg of the drug at the tumor [46]. Moreover, the drug release can be operationally tuned from hours to days to weeks depending on the MSN surface functionalization, thus allowing for the fine adjustment to the different clinical applications and therapeutic regimens.

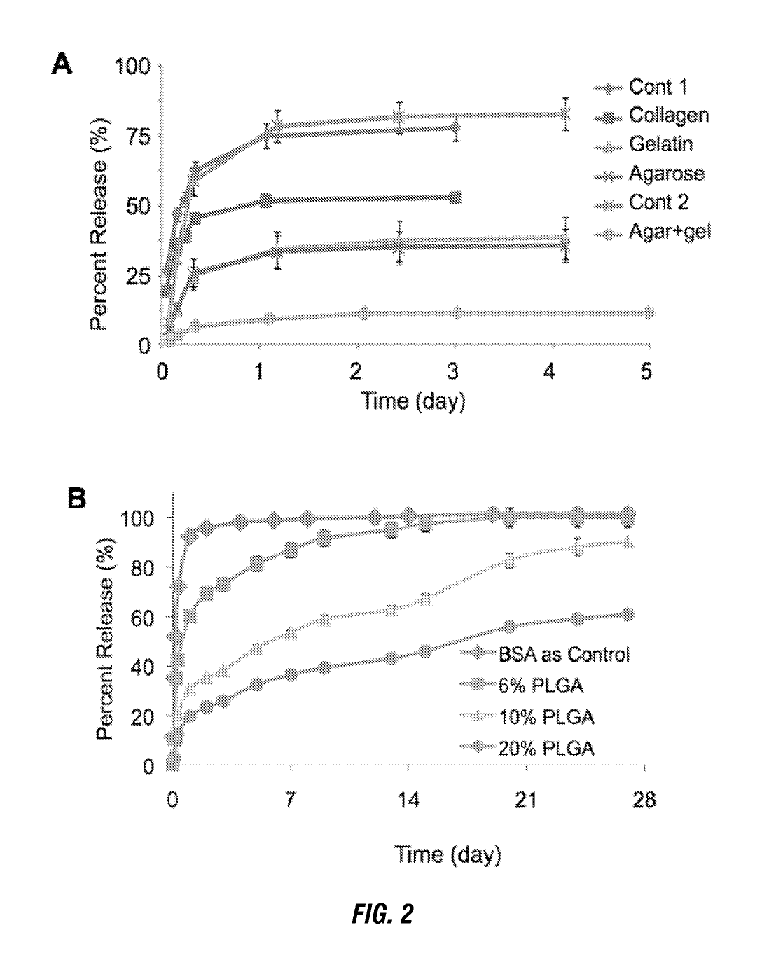

FDA approved polymers (e.g., collagen, gelatin, agarose, poly-lactic-coglycolic acid (PLGA), as well as combinations of them) have been used as coating materials to control drug release from MSN. FIG. 2 shows the release of a model protein (FITC-conjugated bovine serum albumin) from MSN coated with the aforementioned polymers and illustrates the tunable nature of MSN to produce distinct release profiles and rates [31]. Moreover, similar data has been obtained with a diverse panel of biomolecules, including antibiotics, growth factors and differentiating agents. All together, this data confirm that MSN provide an effective way to deliver therapeutic doses of a model chemotheraputic and represent the founding ground for the development of the ASC directed delivery

ASCs are capable of efficiently internalizing a large payload of MSN and showed that MSN are biocompatible with ASC (FIG. 3a,b). We thoroughly characterized MSN subcellular localization and concluded that the uptake of MSN did not induce any significant change in their cytoskeletal structure and overall viability. Moreover, by formulating DOX in micelles and by loading them within the MSN, it was possible to achieve the delayed delivery of a cytotoxic therapeutic in a timely manner. This result is particularly important, as the premature release (or leakage) of the drug would result in the death of the ASC as they migrate to the target (FIG. 3d). Lastly, by injecting ASC/MSN into mice bearing xenograft cancer, we demonstrated with near infrared (NIR) optical imaging, that MSN uptake by ASC did not interfere with their capacity to migrate and home to the tumor mass within four hours (FIG. 3f). Studies indicate that ASC derived from human WAT are as efficient in homing to tumors in mouse xenograft models as allogeneic mouse ASC [39, 47]. In order to assess the translational relevance of this strategy, we are optimizing targeted MSN delivery using human ASC.

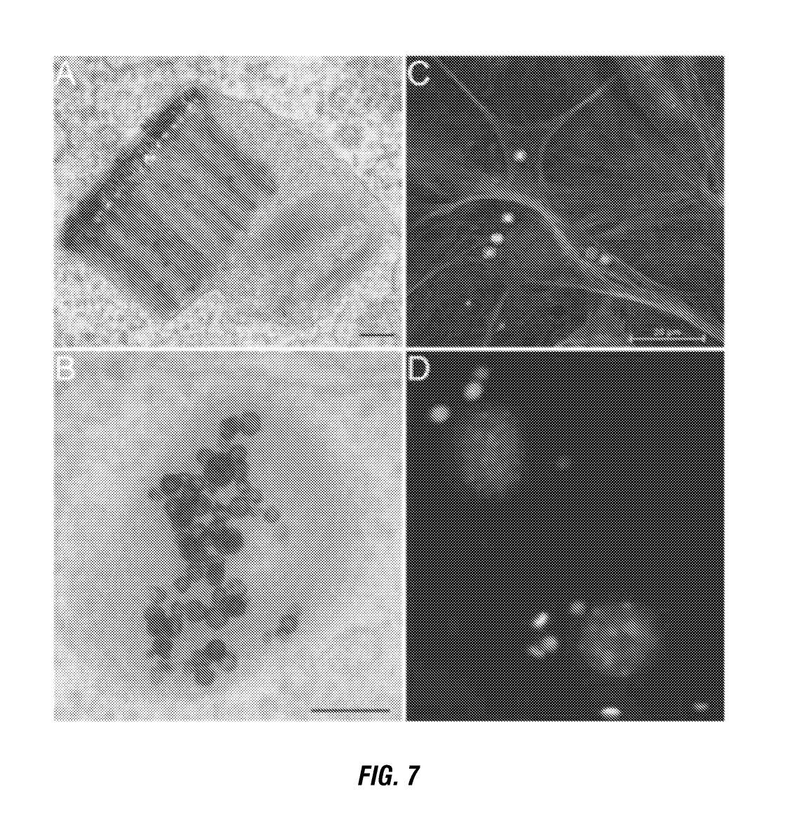

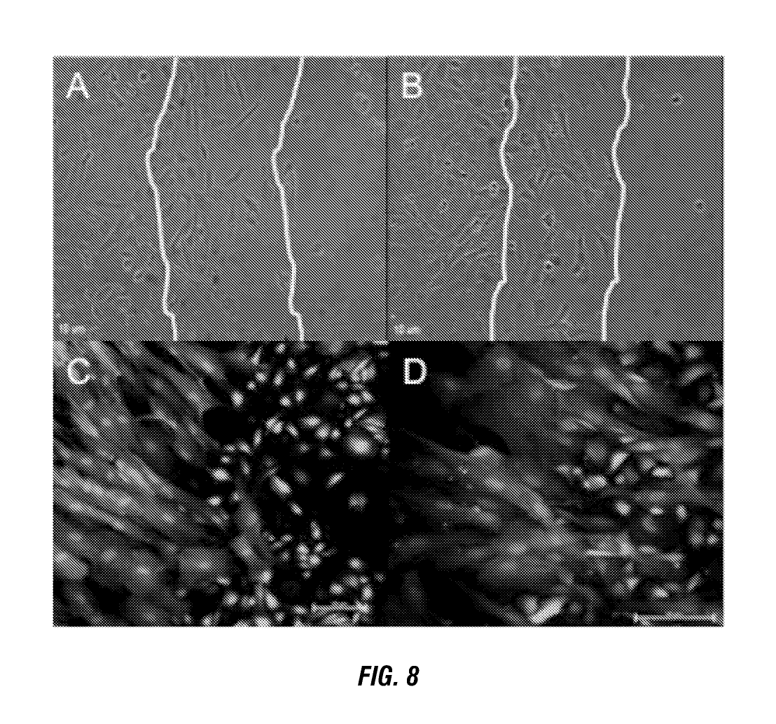

In addition, we have preliminary data confirming that human ASC (hASC) are capable of internalizing multistage particles to a similar extent as was seen with the mouse cells. To supplement our previous data, we have preformed transmission electron microscopy to confirm that hASC can internalize both MSN and gold nanoparticles (AuNPs) with minimal side effects on the cytoskeleton on the cells (FIG. 7). Furthermore, we took advantage of inserts that provide a separation of 500 μm between two compartments that we use to monitor the migration of ASCs towards human breast cancer cells (MDA MB231). Using live microscopy and confocal microscopy, we showed that both mASC and firefly luciferase transduced hASC are capable of migrating towards the cancer cells more so than empty space and a similar rate regardless of the incorporation of MSN (FIG. 8). Furthermore, we proved that the incorporation of MSN does not interfere with the ability of ASCs to differentiate and undergo successful adipogenesis and chondrogenesis.

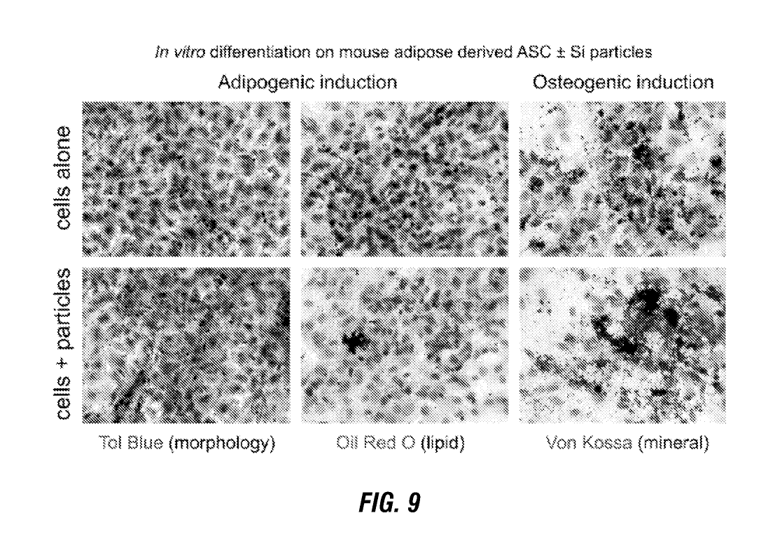

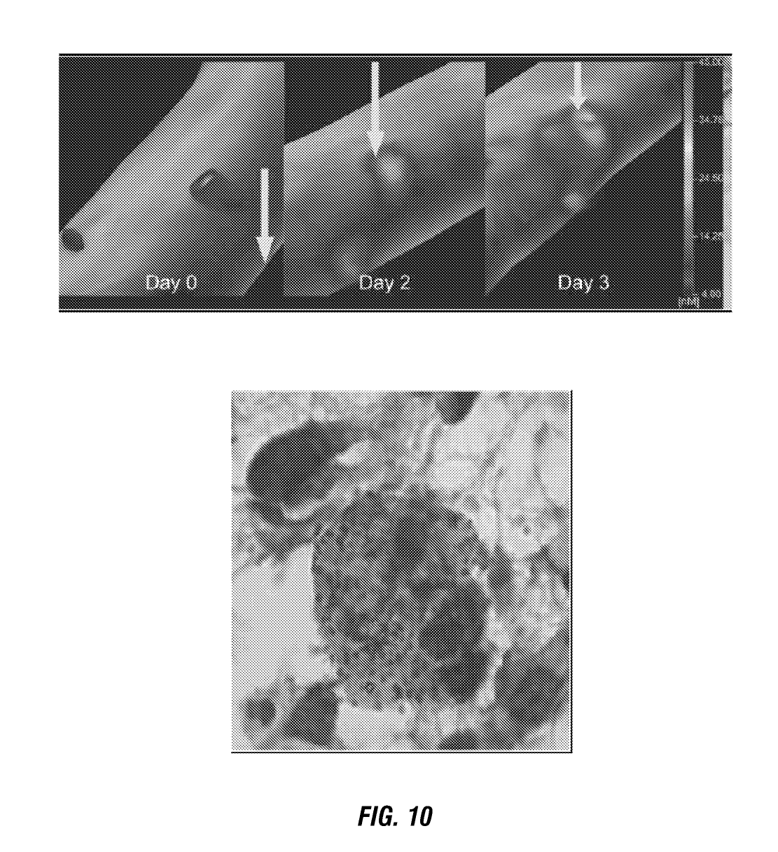

Furthermore, we investigated the ability of ASC-directed vehicles to carry MSN towards a 4T1 orthotopic mouse model of breast cancer. In this experiment, we monitored the migration of ASC by labeling the cells with a NIR dye. We noticed that after allowing the ASC vehicles to migrate for three days a large accumulation of ASCs were seen to migrate towards the tumor site (FIG. 9). After three days, the mice were sacrificed and frozen sections from the tumor were stained with H&E. These tumor sections confirmed the presence of large accumulations of MSN at the tumor site thus providing proof that MSN did not interfere with the tropism of ASCs (FIG. 10).

Radiofrequency is a nonionizing form of energy that can provide local hyperthermia at sites of local accumulation of a variety of nanoparticles (Gannon, Cherukuri et al. 2007; Curley, Cherukuri et al. 2008; Gannon, Patra et al. 2008; Cherukuri and Curley 2010; Cherukuri, Glazer et al. 2010; Glazer and Curley 2010; Glazer, Massey et al. 2010). Preliminary experiments on multistage particles show that heating rates similar to gold nanoparticles (AuNPs) can be achieved at achievable amounts of silicon that can be delivered through this stem cell strategy. ASC show that they can withstand 5 min of RF exposure before displaying any significant cellular abnormalities. Thus, when incorporated with multistage particles, it is expected that ASC will show more sensitivity towards RF (based on the intrinsic heating of multistage loaded with AuNPs) and result not only in the selective destruction of ASC but provide local heat and release of multistage particles specifically in the tumor microenvironment.

Methods

Source of ASC:

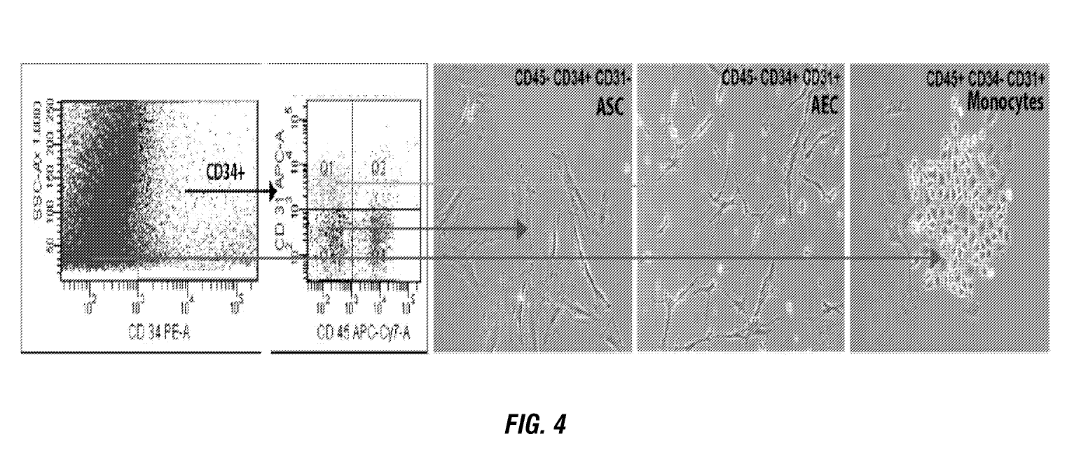

One can use subcutaneous WAT from wild-type mice (C57BL/6 background) ubiquitously expressing GFP from beta-actin promoter [1]. As we have shown, mouse ASC are efficient in tumor homing, and their use in allogeneic tumor models will simulate autologous transplantations projected in the clinical setting. FIG. 4 shows a standard case of ASC isolation from mouse WAT.

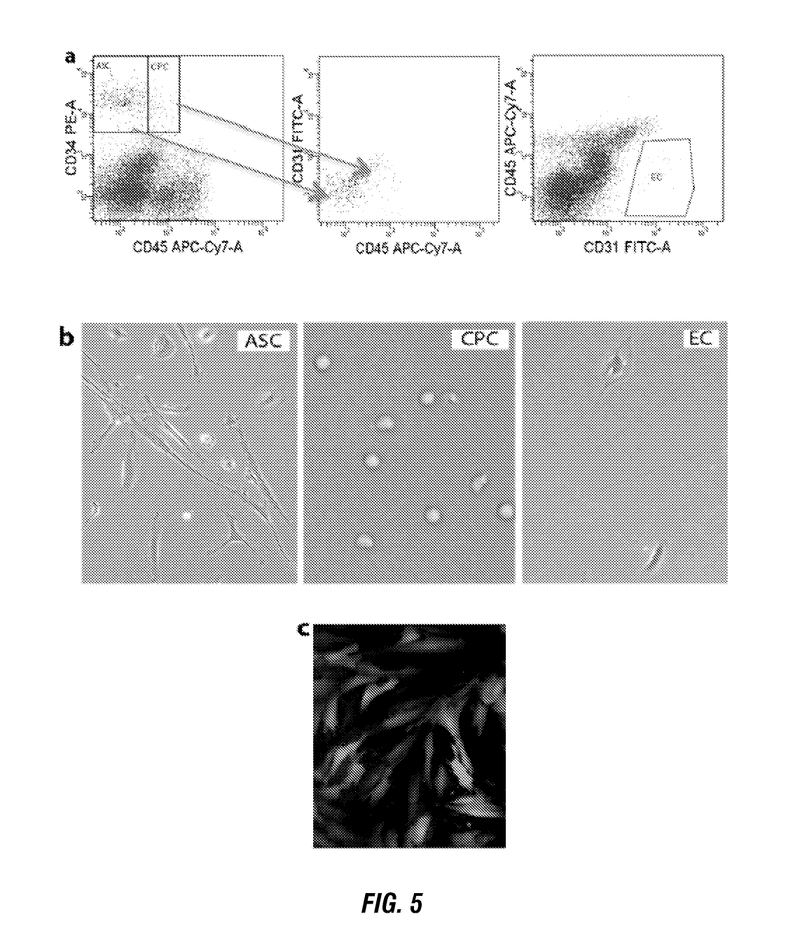

For applications with human tumor xenograft models, human patient subcutaneous WAT-derived ASC can be used. Efficient human ASC migration to human tumors in the animal model is an important advance toward translating our approach. The corresponding dataset demonstrating a routinely performed isolation of ASC from clinical WAT samples is shown in FIG. 5. Both human and mouse ASC can be purified in quantities of at least 10⁶ cells from as little as 25 cc of WAT, which is typical of a clinical specimen easily obtainable by liposuction and is equivalent to the amount of WAT derived from 10-15 C57BL/6 mice raised on high fat diet and rendered obese, as we have previously shown [1, 48]. Because ASC typically proliferate rapidly for the first 6 passages, this yield allows one to easily produce 10⁷ to 10⁸ low-passage cells within a week of isolation from WAT [43], which is more than sufficient for experiments proposed. This demonstrates the viability of our approach for applications with autologous ASC isolated from dogs as previously described [49] and from patients in subsequent clinical studies.

MSN Fabrication:

Hemispherical MSN with a diameter of 950 nm to 3200 nm and pore sizes between 10 nm to 100 nm are fabricated. In addition, one can fabricate discoidal MSN with diameters ranging from 600 nm to 2600 nm, with pore sizes between 20 nm to 100 nm. Briefly, MSNs are fabricated by combining nanolithography and electro-chemical porosification of silicon in a HF solution. The size and shape of MSN will be defined by photolithography, yielding monodispersed MSN with precisely controlled size and shapes [50].

In order to achieve the full potential of the theranostic approach providing imaging and therapy in one platform, one can harness the capacity of MSN to incorporate both imaging and therapeutic NP. Due to the flexibility of use and versatility of MSN features, several approaches are available including the conjugation of dyes to its surface and the simultaneous loading of NIR NP or metallic NP (e.g., SPIO, gold) with the therapeutic agent. The potential of this approach can be evaluated in tumor models and the efficacy will be determined as described above.

Statistical Analysis

Statistical analysis will be performed with sufficient power for the desired inference goals and for data analysis upon completion of every experiment. Categorical data (response to treatment, cancer remission) will be described using contingency tables, and we will test for differences among treatment or cohort groups by using Pearson's chi-square statistics. Continuously scaled measures will be summarized with descriptive statistical measures (i.e., mean (±s.d.) and median (range)). For the analysis of continuous variables (tumor size, % of cells death, etc), we will use general linear models, analysis of variance (ANOVA), covariance (ANCOVA) and Student's T-test. Differences will be considered significant for p values less than 0.05.

Methods

The following methods can be used to promote the release of the payload upon reaching the target site:

NP Coatings to Promote Endosome/Lysosome Escape

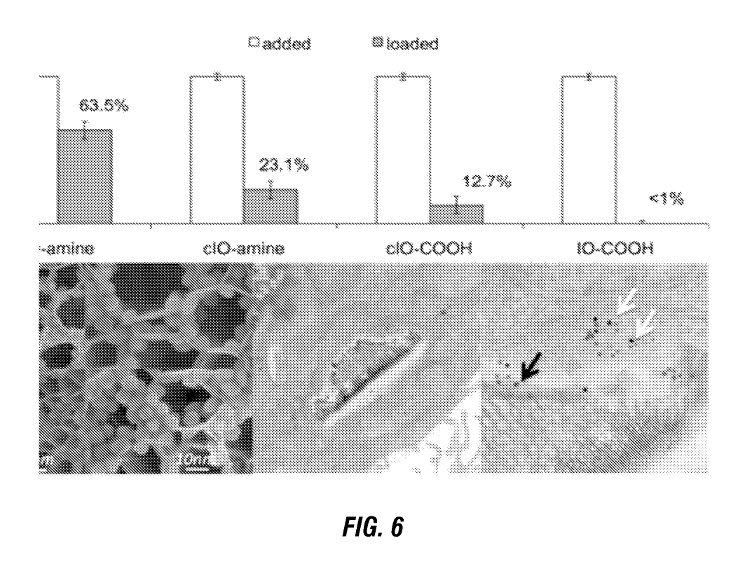

Chitosan-coated iron oxide (cIO) NP were efficiently loaded into MSN (FIG. 6). TEM was used to visualize the localization of these 30 nm NP following cell internalization. Endosomal escape, characterized by the presence of free NP in the cytoplasm, was limited to cIO particles delivered by MSN. Unmodified NP, which expressed amine or carboxyl groups, remained inside the endosomes for up to 7 days. The use of the MSN is critical to time the triggering of NP delivery as it provides the benefit of protecting the chitosan from the signals that would trigger its premature release. This preliminary data suggest that controlled endosomal escape of NP from MSN within ASC allows for the timed release of the cytotoxic payload after ASC tumor engraftment.

Swelling of Ionic Hydrogels

Smart hydrogels can swell or shrink with changes in external conditions such as pH and temperature. Herein, we will take advantage of the pH changes that occurs in endosomes (lower pH, ˜4-6) and produce hydrogels that swell at pHs lower than 7. The surface modification of MSN with hydrogels can be tuned so that, upon internalization, swelling occurs after a few hours or a few days allowing sufficient time for the ASC to home to the tumor. The induced swelling of the hydrogel coating within the lysosome will result in the vesicle's disruption. The leakage of enzymes and ionic species in the cytoplasm would lead to the necrotic death of ASC and to the release of MSN/NP in the tumor microenvironment.

Thermal Destruction of ASC

The thermal heating of nanoparticles upon exposure with radiofrequency (RF), magnetic fields and near infrared (NIR) lasers have shown promise for noninvasive local hyper-thermal therapy. Magnetic field and NIR excitation are typically achieved using iron oxide and appropriate forms of gold (rods, shells, hollow spheres), respectfully. However RF has shown promise over a broad range of nanoparticles including, but certainly not limited to: gold, quantum dots, carbon nanotubes, silver, iron oxide, and silicon nanoparticles. All three hyperthermia methods provide minimal side-effects to tissues that do not contain large accumulations and will provide local, concentrated hyperthermia.

The versatility of the multistage platform allows us to incorporate a variety of payloads in order to achieve the previously mentioned forms of thermal heating. We also believe that other forms of heating may be beneficial in different settings. In addition, rather than relying on MSN to provide payloads for specific heating, we can allow ASC to incorporate benign payloads separate from MSN that will provide added benefit. Hence, we will be able to use multiple forms of excitation for thermal therapy as well as simultaneous excitation if deemed advantageous.

Induced Necrosis of ASC Upon Reaching Tumor Site

One can pre-treat the cells with agents that allow the cells to migrate, however upon homing to the target site they selectively become activated and destroy the ASC carrier. Alternatively, ASC can be treated with nanoparticles that can control the release of agents which results in the burst release of agents that result in the rapid destruction of the ASC releasing the payload at the site of infection.

### REFERENCES

1. Zhang Y, Daquinag A, Traktuev D O, Amaya F, Simmons P J, March K L, et al. White adipose tissue cells are recruited by experimental tumors and promote cancer progression in mouse models. Cancer Res 2009;69(12):5259-5266.
2. Tasciotti E, Liu X, Bhavane R, Plant K, Leonard A D, Price B K, et al. Mesoporous silicon particles as a multistage delivery system for imaging and therapeutic applications. Nat Nanotechnol 2008 March;3(3):151-157.
3. Saga T, Koizumi M, Furukawa T, Yoshikawa K, Fujibayashi Y. Molecular imaging of cancer: evaluating characters of individual cancer by PET/SPECT imaging. Cancer Sci 2009 March;100(3):375-381.
4. Ferrari M. Cancer nanotechnology: opportunities and challenges. Nat Rev Cancer 2005 March;5(3):161-171.
5. Zhang L, Gu F X, Chan J M, Wang A Z, Langer R S, Farokhzad O C. Nanoparticles in medicine: therapeutic applications and developments. Clin Pharmacol Ther 2008 May;83(5):761-769.
6. Ferrari M. Nanovector therapeutics. Curr Opin Chem Biol 2005 August;9(4):343-346.
7. Peer D, Karp J M, Hong S, Farokhzad O C, Margalit R, Langer R. Nanocarriers as an emerging platform for cancer therapy. Nat Nanotechnol 2007 December;2(12):751-760.
8. Decuzzi P, Pasqualini R, Arap W, Ferrari M. Intravascular delivery of particulate systems: does geometry really matter? Pharm Res 2009 January;26(1):235-243.
9. Debbage P. Targeted drugs and nanomedicine: present and future. Curr Pharm Des 2009;15(2):153-172.
10. Decuzzi P, Lee S, Decuzzi M, Ferrari M. Adhesion of microfabricated particles on vascular endothelium: a parametric analysis. Ann Biomed Eng 2004 June;32(6):793-802.
11. Lee S Y, Ferrari M, Decuzzi P. Shaping nano-/micro-particles for enhanced vascular interaction in laminar flows. Nanotechnology 2009 Dec. 9;20(49):495101.
12. Decuzzi P, Godin B, Tanaka T, Lee S Y, Chiappini C, Liu X, et al. Size and shape effects in the biodistribution of intravascularly injected particles. J Control Release 2010 Feb. 15;141(3):320-327.
13. Lee S Y, Ferrari M, Decuzzi P. Design of bio-mimetic particles with enhanced vascular interaction. J Biomech 2009 Aug. 25;42(12):1885-1890.
14. Gentile F, Curcio A, Indolfi C, Ferrari M, Decuzzi P. The margination propensity of spherical particles for vascular targeting in the microcirculation. J Nanobiotechnology 2008;6:9.
15. Gentile F, Chiappini C, Fine D, Bhavane R C, Peluccio M S, Cheng M M, et al. The effect of shape on the margination dynamics of non-neutrally buoyant particles in two-dimensional shear flows. J Biomech 2008 Jul. 19;41(10):2312-2318.
16. Gentile F, Ferrari M, Decuzzi P. The transport of nanoparticles in blood vessels: the effect of vessel permeability and blood rheology. Ann Biomed Eng 2008 February;36(2):254-261.
17. Decuzzi P, Ferrari M. The receptor-mediated endocytosis of nonspherical particles. Biophys J 2008 May 15;94(10):3790-3797.
18. Decuzzi P, Ferrari M. Design maps for nanoparticles targeting the diseased microvasculature. Biomaterials 2008 January;29(3):377-384.
19. Decuzzi P, Ferrari M. The adhesive strength of non-spherical particles mediated by specific interactions. Biomaterials 2006 October;27(30):5307-5314.
20. Canham L. Bioactive silicon structure fabrication through nanoetching techniques. Advanced Materials 1995;7(12):1033-1037.
21. Canham L, Reeves C L, Newey J, Houlton M, Cox T, Buriak J, et al. Derivatized Mesoporous Silicon with Dramatically Improved Stability in Simulated Human Blood Plasma. Advanced Materials 1999;11(18):1505-1507.
22. Canham L T, INSPEC (Information service), Institution of Electrical Engineers. Properties of porous silicon. London: IEE INSPEC, 1997.
23. Chiappini C, Tasciotti E, Fakhoury J, Fine D. Pullan L, Wang Y, et al. Tailored porous silicon microparticles: fabrication and properties. Chem Phys Chem 2010.
24. Sakamoto J, Annapragada A. Decuzzi P, Ferrari M. Antibiological barrier nanovector technology for cancer applications. Expert Opin Drug Deliv 2007 July;4(4):359-369.
25. van de Ven A L, Serda R E, Mack A C, Ferrati S, Godin B, Ferrari M. Intracellular partitioning and exosomal release of nanoparticles. In preparation 2010.
26. Tanaka T, Mangala L S, Vivas-Mejia P E, Nieves R A, Mann A P, Mora E, et al. Sustained siRNA Delivery by Mesoporous Silicon Particles for Cancer Treatment. Cancer Res 2010; In press.
27. Serda R E, Ferrati S, Godin B, Tasciotti E, Liu X, Ferrari M. Mitotic trafficking of silicon microparticles. Nanoscale 2009;1(2):250-259.
28. Salonen J, Laitinen L, Kaukonen A M, Tuura J, Bjorkqvist M, Heikkila T, et al. Mesoporous silicon microparticles for oral drug delivery: loading and release of five model drugs. J Control Release 2005 Nov. 28;108(2-3):362-374.
29. Anglin E J, Schwartz M P, Ng V P, Perelman L A, Sailor M J. Engineering the chemistry and nanostructure of porous silicon Fabry-Perot films for loading and release of a steroid. Langmuir 2004 Dec. 7;20(25):11264-11269.
30. Prestidge C A, Barnes T J, Mierczynska-Vasilev A, Kempson I, Peddie F, Barnett C. Peptide and protein loading into porous silicon wafer. Phys Stat Sol 2008;205(2):311-315.
31. Fan D, Peng Y, Smid C A, Chiappini C, Murphy M, Liu X, et al. Nano-Structured Silicon/PLGA Composite Microspheres for the Sustained Release of Biomolecules. In preparation 2010.
32. Martinez J O, Chiappini C, Liu X, Ferrari M. Tasciotti E. Nanoporous silicon particles with tailored characteristics for controlled biodegradation. In preparation 2010.
33. Dvorak H F. Tumors: wounds that do not heal. Similarities between tumor stroma generation and wound healing. N Engl J Med 1986 Dec. 25;315(26):1650-1659.
34. Bianco P, Robey P G, Simmons P J. Mesenchymal stem cells: revisiting history, concepts, and assays. Cell Stem Cell 2008 Apr. 10;2(4):313-319.
35. Caplan A I. Adult mesenchymal stem cells for tissue engineering versus regenerative medicine. J Cell Physiol 2007 November;213(2):341-347.
36. Prockop D J. Repair of tissues by adult stem/progenitor cells (MSCs): controversies, myths, and changing paradigms. Mol Ther 2009 June;17(6):939-946.
37. Lazennec G, Jorgensen C. Concise review: adult multipotent stromal cells and cancer: risk or benefit? Stem Cells 2008 June;26(6):1387-1394.
38. Kolonin M G, Simmons P J. Combinatorial stem cell mobilization Nat Biotechnol 2009;27(3):252-253.
39. Kucerova L, Altanerova V, Matuskova M, Tyciakova S, Altaner C. Adipose tissue-derived human mesenchymal stem cells mediated prodrug cancer gene therapy. Cancer Res 2007 Jul. 1;67(13):6304-6313.
40. Studeny M, Marini F C, Dembinski J L, Zompetta C, Cabreira-Hansen M, Bekele B N, et al. Mesenchymal stem cells: potential precursors for tumor stroma and targeted-delivery vehicles for anticancer agents. J Natl Cancer Inst 2004 Nov. 3;96(21):1593-1603.
41. Hall B, Dembinski J, Sasser A K, Studeny M, Andreeff M, Marini F. Mesenchymal stem cells in cancer: tumor-associated fibroblasts and cell-based delivery vehicles. Int J Hematol 2007 July;86(1):8-16.
42. Wu X, Hu J, Zhou L. Mao Y, Yang B, Gao L, et al. In vivo tracking of superparamagnetic iron oxide nanoparticle-labeled mesenchymal stem cell tropism to malignant gliomas using magnetic resonance imaging. Laboratory investigation. J Neurosurg 2008 February;108(2):320-329.
43. Traktuev D, Merfeld-Clauss S, Li J, Kolonin M, Arap W, Pasqualini R, et al. A Population of multipotent CD34-positive adipose stromal cells share pericyte and mesenchymal surface markers, reside in a periendothelial location, and stabilize endothelial networks. Circ Res 2008 Jan. 4;102(1):77-85.
44. Nie J, Chang B, Traktuev D O, Sun J, March K L, Chan L, et al. Combinatorial peptides identify α5β1 integrin as a receptor for the matricellular protein SPARC on adipose stromal cells. Stem Cells 2008;26(10):2735-2745.
45. Kolonin M G. Tissue-specific targeting based on markers expressed outside endothelial cells. Adv Genet 2009;67:61-102.
46. Xiong X B, Huang Y, Lu W L, Zhang H, Zhang X, Zhang Q. Enhanced intracellular uptake of sterically stabilized liposomal Doxorubicin in vitro resulting in improved antitumor activity in vivo. Pharm Res 2005 June;22(6):933-939.
47. Muehlberg F, Song Y H, Krohn A, Pinilla S, Droll L, Leng X, et al. Tissue resident stem cells promote breast cancer growth and metastasis. Carcinogenesis 2009 Jan. 30.
48. Kolonin M G, Saha P K, Chan L, Pasqualini R, Arap W. Reversal of obesity by targeted ablation of adipose tissue. Nat Med 2004 June;10(6):625-632.
49. Lee B J, Wang S G, Lee J C, Jung J S, Bae Y C, Jeong H J, et al. The prevention of vocal fold scarring using autologous adipose tissue-derived stromal cells. Cells Tissues Organs 2006; 184(3-4): 198-204.
50. Chiappini C, Tasciotti E, Fakhoury J, Fine D. Pullan L, Wang Y, et al. Tailored porous silicon microparticles: fabrication and properties. Chem Phys Chem 2010.
51. Tang N, Du G, Wang N, Liu C, Hang H, Liang W. Improving penetration in tumors with nanoassemblies of phospholipids and doxorubicin. J Natl Cancer Inst 2007 Jul. 4;99(13):1004-1015.
52. Stavridi F, Palmieri C. Efficacy and toxicity of nonpegylated liposomal doxorubicin in breast cancer. Expert Rev Anticancer Ther 2008 December;8(12):1859-1869.
53. Verma S, Dent S, Chow B J, Rayson D, Safra T. Metastatic breast cancer: the role of pegylated liposomal doxorubicin after conventional anthracyclines. Cancer Treat Rev 2008 Aug;34(5):391-406.
54. Gabizon A A. Pegylated liposomal doxorubicin: metamorphosis of an old drug into a new form of chemotherapy. Cancer Invest 2001;19(4):424-436.
55. Tanaka T, Decuzzi P, Cristofanilli M, Sakamoto J H, Tasciotti E, Robertson F M, et al. Nanotechnology for breast cancer therapy. Biomed Microdevices 2009 February;11(1):49-63.
56. Sakamoto J H, van de Ven A L, Godin B, Blanco E, Serda R E, Grattoni A, et al. Enabling individualized therapy through nanotechnology. Pharmacol Res 2010 Jan. 4.
57. Frank J A, Miller B R, Arbab A S, Zywicke H A, Jordan E K, Lewis B K, et al. Clinically applicable labeling of mammalian and stem cells by combining superparamagnetic iron oxides and transfection agents. Radiology 2003 Aug;228(2):480-487.
58. Ferrati S, Mack A C, Bean A, Serda R E, Ferrari M. Intracellular trafficking of silicon nanocarriers. In preparation 2010.
59. Fisher O Z, Kim T, Dietz S R, Peppas N A. Enhanced core hydrophobicity, functionalization and cell penetration of polybasic nanomatrices. Pharm Res 2009 January;26(1):51-60.
60. Serra L, Domenech J, Peppas N A. Drug transport mechanisms and release kinetics from molecularly designed poly(acrylic acid-g-ethylene glycol) hydrogels. Biomaterials 2006 November;27(31):5440-5451.
61. Hirsch L R, Stafford R J, Bankson J A, Sershen S R, Rivera B, Price R E, et al. Nanoshell-mediated near-infrared thermal therapy of tumors under magnetic resonance guidance. Proc Natl Acad Sci U S A 2003 Nov. 11;100(23):13549-13554.
62. Gobin A M, Lee M H, Halas N J, James W D, Drezek R A, West J L. Near-infrared resonant nanoshells for combined optical imaging and photothermal cancer therapy. Nano Lett 2007 July;7(7):1929-1934.
63. Tasciotti E, Giacca M. Fusion of the human immunodeficiency virus type 1 tat protein transduction domain to thymidine kinase increases bystander effect and induces enhanced tumor killing in vivo. Hum Gene Ther 2005 December;16(12):1389-1403.
64. De Coupade C, Fittipaldi A, Chagnas V, Michel M, Carlier S, Tasciotti E, et al. Novel human-derived cell-penetrating peptides for specific subcellular delivery of therapeutic biomolecules. Biochem J 2005 Sep. 1;390(Pt 2):407-418.
65. Tasciotti E, Zoppe M, Giacca M. Transcellular transfer of active HSV-1 thymidine kinase mediated by an 11-amino-acid peptide from HIV-1 Tat. Cancer Gene Ther 2003 January;10(1):64-74.
66. Sawant R R, Torchilin V P. Enhanced cytotoxicity of TATp-bearing paclitaxel-loaded micelles in vitro and in vivo. Int J Pharm 2009 Jun. 5;374(1-2):114-118.
67. Torchilin V P. Cell penetrating peptide-modified pharmaceutical nanocarriers for intracellular drug and gene delivery. Biopolymers 2008;90(5):604-610.
68. Santra S, Yang H, Dutta D, Stanley J T, Holloway P H, Tan W, et al. TAT conjugated, FITC doped silica nanoparticles for bioimaging applications. Chem Commun (Camb) 2004 Dec. 21(24):2810-2811.
69. Bassukas I D, Maurer-Schultze B. Relationship between preimplantation host weight and growth of the mouse adenocarcinoma EO 771. In Vivo 1992 January-February;6(1):93-96.
70. Kidd S, Spaeth E, Dembinski J L, Dietrich M, Watson K, Klopp A, et al. Direct evidence of mesenchymal stem cell tropism for tumor and wounding microenvironments using in vivo bioluminescent imaging. Stem Cells 2009 October;27(10):2614-2623.
71. Spaeth E L, Dembinski J L, Sasser A K, Watson K, Klopp A, Hall B, et al. Mesenchymal stem cell transition to tumor-associated fibroblasts contributes to fibrovascular network expansion and tumor progression. PLoS One 2009;4(4):e4992.
72. Vilalta M, Degano I R, Bago J, Gould D, Santos M. Garcia-Arranz M, et al. Biodistribution, long-term survival, and safety of human adipose tissue-derived mesenchymal stem cells transplanted in nude mice by high sensitivity non-invasive bioluminescence imaging. Stem Cells Dev 2008 October;17(5):993-1003.
73. Ferrari M. Cancer nanotechnology: opportunities and challenges. Nat Rev Cancer 2005 March;5(3):161-171.
74. Darzynkiewicz Z, Bruno S, Del Bino G, Gorczyca W, Hotz M A, Lassota P. et al. Features of apoptotic cells measured by flow cytometry. Cytometry 1992;13(8):795-808.
75. Arap W, Pasqualini R, Ruoslahti E. Cancer treatment by targeted drug delivery to tumor vasculature in a mouse model. Science 1998;279 (5349):377-380.
76. Grimes B R, Steiner C M, Merfeld-Clauss S, Traktuev D O, Smith D, Reese A, et al. Interphase FISH demonstrates that human adipose stromal cells maintain a high level of genomic stability in long-term culture. Stem Cells Dev 2009 June;18(5):717-724.
77. Cherukuri, P. and S. A. Curley (2010). “Use of nanoparticles for targeted, noninvasive thermal destruction of malignant cells.” Methods Mol Biol 624: 359-373.
78. Cherukuri, P., E. S. Glazer, et al. (2010). “Targeted hyperthermia using metal nanoparticles.” Adv Drug Deliv Rev 62(3): 339-345.
78. Curley, S. A., P. Cherukuri, et al. (2008). “Noninvasive radiofrequency field-induced hyperthermic cytotoxicity in human cancer cells using cetuximab-targeted gold nanoparticles.” J Exp Ther Oncol 7(4): 313-326.
79. Gannon, C. J., P. Cherukuri, et al. (2007). “Carbon nanotube-enhanced thermal destruction of cancer cells in a noninvasive radiofrequency field.” Cancer 110(12): 2654-2665. Gannon, C. J., C. R. Patra, et al. (2008). “Intracellular gold nanoparticles enhance non-invasive radiofrequency thermal destruction of human gastrointestinal cancer cells.” J Nanobiotechnology 6:2.
80. Gimble, J. M., A. J. Katz, et al. (2007). “Adipose-derived stem cells for regenerative medicine.” Circ Res 100(9): 1249-1260.
81. Glazer, E. S. and S. A. Curley (2010). “Radiofrequency field-induced thermal cytotoxicity in cancer cells treated with fluorescent nanoparticles.” Cancer 116(13): 3285-3293. Glazer, E. S., K. L. Massey, et al. (2010). “Pancreatic carcinoma cells are susceptible to noninvasive radio frequency fields after treatment with targeted gold nanoparticles.” Surgery 148(2): 319-324.
82. Jeong, J. H. (2010). “Adipose stem cells and skin repair.” Current stem cell research & therapy 5(2): 137-140.
83. Kim, W. S., B. S. Park, et al. (2007). “Wound healing effect of adipose-derived stem cells: a critical role of secretory factors on human dermal fibroblasts.” Journal of dermatological science 48(1): 15-24.
84. Libby, P., P. M. Ridker, et al. (2009). “Inflammation in atherosclerosis: from pathophysiology to practice.” J Am Coll Cardiol 54(23): 2129-2138.
85. Sahaf, B., K. Heydari, et al. (2003). “Lymphocyte surface thiol levels.” Proceedings of the National Academy of Sciences of the United States of America 100(7): 4001-4005.
86. Stephan, M. T., J. J. Moon, et al. (2010). “Therapeutic cell engineering with surface-conjugated synthetic nanoparticles.” Nature medicine 16(9): 1035-1041.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A modified stem cell comprising:
a delivery system,
wherein the delivery system comprises at least one microparticle or nanoparticle, and
wherein the at least one microparticle or nanoparticle comprises an active agent.

2. The modified stem cell of claim 1, wherein the at least one microparticle or nanoparticle comprises a porous particle.

3. The modified stem cell of claim 2, wherein the porous particle comprises at least one of a nanoporous silicon particle or a nanoporous silica particle.

4. The modified stem cell of claim 1, wherein the at least one microparticle or nanoparticle is selected from the group consisting of multistage particles, porous particles, porous silicon particles, porous silica particles, non-porous particles, fabricated particles, polymeric particles, synthetic particles, semiconducting particles, viruses, gold particles, silver particles, quantum dots, indium phosphate particles, iron oxide particles, micelles, liposomes, silica particles, mesoporous silica particles, PLGA-based particles, gelatin-based particles, carbon nanotubes, fullerenes, and combinations thereof.

5. The modified stem cell of claim 1, wherein the at least one microparticle or nanoparticle comprises a particle with a functionalized surface.

6. The modified stem cell of claim 5, wherein the surface of the particle is functionalized with a functionalizing agent selected from the group consisting of peptides, polymers, chitosans, contrasting agents, imaging agents and calcium phosphates.

7. The modified stem cell of claim 5, wherein the surface of the particle is functionalized with a polymer, wherein the polymer becomes swellable in response to a stimulus selected from the group consisting of change in temperature, change in pH, change in pressure, and combinations thereof.

8. The modified stem cell of claim 1, wherein the active agent comprises a therapeutic agent.

9. The modified stem cell of claim 8, wherein the therapeutic agent is selected from the group consisting of anti-inflammatory agents, anti-cancer agents, anti-proliferative agents, anti-vascularization agents, wound repair agents, tissue repair agents, thermal therapy agents, and combinations thereof.

10. The modified stem cell of claim 1, wherein the active agent comprises an imaging agent.

11. The modified stem cell of claim 1, wherein the modified stem cell is selected from the group consisting of adult stem cells, embryonic stem cells, fetal stem cells, mesenchymal stem cells, neural stem cells, totipotent stem cells, pluripotent stem cells, multipotent stem cells, oligopotent stem cells, unipotent stem cells, adipose stromal cells, endothelial stem cells, and combinations thereof.

12. The modified stem cell of claim 1, wherein the modified stem cell is an adipose stromal stem cell.

13. The modified stem cell of claim 1, wherein the delivery system is a multistage delivery system.

14. The modified stem cell of claim 1, wherein the delivery system is on a surface of the modified stem cell.

15. The modified stem cell of claim 1, wherein the delivery system is inside the modified stem cell.

16. The modified stem cell of claim 1, wherein the modified stem cell is used to treat, monitor, diagnose, or prevent a condition associated with inflammation.

17. The modified stem cell of claim 14, wherein the condition to be treated, monitored, diagnosed, or prevented is cancer.

18. A delivery method comprising:
administering to a subject a modified stem cell comprising:
a delivery system, wherein the delivery system comprises at least one microparticle or nanoparticle, and
wherein the at least one microparticle or nanoparticle comprises an active agent.

19. The delivery method of claim 18, wherein the administering comprises at least one of intravenous administration, subcutaneous administration, and intramuscular administration.

20. The delivery method of claim 18, wherein the modified stem cell is selected from the group consisting of adult stem cells, embryonic stem cells, fetal stem cells, mesenchymal stem cells, neural stem cells, totipotent stem cells, pluripotent stem cells, multipotent stem cells, oligopotent stem cells, unipotent stem cells, adipose stromal cells, endothelial stem cells, and combinations thereof.

21. The delivery method of claim 18, wherein the modified stem cell is derived directly from the subject.

22. The delivery method of claim 18, wherein the subject is a human being suffering from a condition.

23. The delivery method of claim 22, wherein the condition is associated with inflammation.

24. The delivery method of claim 23, wherein the condition associated with inflammation is cancer.

25. The delivery method of claim 22, wherein the modified stem cell migrates to a site associated with the condition within the subject after administration.

26. The delivery method of claim 25, wherein the active agent is released from the modified stem cell after migration to the site associated with the condition.

27. The delivery method of claim 26, wherein the release of the active agent from the modified stem cell comprises lysis of the modified stem cells.

28. The delivery method of claim 27, further comprising inducing the lysis of the modified stem cells by exposing the stem cells to a stimulus selected from the group consisting of radiofrequency signals, heat, magnetic field radiation, light, changes in pressure, changes in pH, changes in temperature and combinations thereof.

29. The delivery method of claim 26, wherein the release of the active agent from the modified stem cell comprises time-dependent necrosis of the modified stem cells.

30. The delivery method of claim 18, wherein the active agent comprises a therapeutic agent selected from the group consisting of anti-inflammatory agents, anti-cancer agents, anti-proliferative agents, anti-vascularization agents, wound repair agents, tissue repair agents, thermal therapy agents, and combinations thereof.

31. The delivery method of claim 18, wherein the active agent comprises an imaging agent.

32. The delivery method of claim 18, wherein the at least one microparticle or nanoparticle is selected from the group consisting of multistage particles, porous particles, porous silicon particles, porous silica particles, non-porous particles, fabricated particles, polymeric particles, synthetic particles, semiconducting particles, viruses, gold particles, silver particles, quantum dots, indium phosphate particles, iron oxide particles, micelles, liposomes, silica particles, mesoporous silica particles, PLGA-based particles, gelatin-based particles, carbon nanotubes, fullerenes, and combinations thereof.

33. The delivery method of claim 18, wherein the at least one microparticle or nanoparticle comprises a particle with a functionalized surface, wherein the surface is functionalized with a functionalizing agent selected from the group consisting of peptides, polymers, chitosans, contrasting agents, imaging agents and calcium phosphates.

34. The delivery method of claim 18, wherein the delivery system is on a surface of the modified stem cell.

35. The delivery method of claim 18, wherein the delivery system is inside the modified stem cell.

36. A method of modifying a stem cell, wherein the method comprises:
associating a delivery system with the stem cell, wherein the delivery system comprises at least one microparticle or nanoparticle, and
wherein the at least one microparticle or nanoparticle comprises an active agent.

37. The method of claim 36, wherein the associating comprises introducing the delivery system inside the stem cell.

38. The method of claim 36, wherein the associating comprises conjugating the delivery system onto a surface of the stem cell.

39. The method of claim 38, wherein the conjugating is facilitated by one or more functional groups on a surface of the delivery system.

40. The method of claim 36, wherein the associating comprises incubating the delivery system with the stem cell.

41. The method of claim 36, wherein the at least one microparticle or nanoparticle is selected from the group consisting of multistage particles, porous particles, porous silicon particles, porous silica particles, non-porous particles, fabricated particles, polymeric particles, synthetic particles, semiconducting particles, viruses, gold particles, silver particles, quantum dots, indium phosphate particles, iron oxide particles, micelles, liposomes, silica particles, mesoporous silica particles, PLGA-based particles, gelatin-based particles, carbon nanotubes, fullerenes. and combinations thereof.

42. The method of claim 36, wherein the at least one microparticle or nanoparticle comprises a particle with a functionalized surface.

43. The method of claim 42, wherein the surface of the particle is functionalized with a functionalizing agent selected from the group consisting of peptides, polymers, chitosans, contrasting agents, imaging agents and calcium phosphates.

44. The method of claim 42, wherein the surface of the particle is functionalized with a polymer, wherein the polymer becomes swellable in response to a stimulus selected from the group consisting of change in temperature, change in pH, change in pressure, and combinations thereof.

45. The method of claim 36, wherein the active agent comprises a therapeutic agent.

46. The method of claim 45, wherein the therapeutic agent is selected from the group consisting of anti-inflammatory agents, anti-cancer agents, anti-proliferative agents, anti-vascularization agents, wound repair agents, tissue repair agents, thermal therapy agents, and combinations thereof.

47. The method of claim 36, wherein the active agent comprises an imaging agent.

48. The method of claim 36, wherein the stem cell is selected from the group consisting of adult stem cells, embryonic stem cells, fetal stem cells, mesenchymal stem cells, neural stem cells, totipotent stem cells, pluripotent stem cells, multipotent stem cells, oligopotent stem cells, unipotent stem cells, adipose stromal cells, endothelial stem cells, and combinations thereof.

49. The method of claim 36, wherein the stem cell is an adipose stromal stem cell.

50. The method of claim 36, wherein the delivery system is a multistage delivery system.

51. The method of claim 36, wherein the modified stem cell is used to treat, monitor, diagnose, or prevent a condition associated with inflammation.

52. The method of claim 51, wherein the condition to be treated, monitored, diagnosed, or prevented is cancer.
